(19) United States
(12) Patent Application Publication
(10) Pub. No.: US 2021/0322462 A1
(43) Pub. Date: Oct. 21, 2021

(54) WOUND PREVENTION AND/OR TREATMENT AND RELATED COMPOUNDS, MATRICES, COMPOSITIONS, METHODS AND SYSTEMS

(71) Applicants: CALIFORNIA INSTITUTE OF TECHNOLOGY, Pasadena, CA (US); THE REGENTS OF THE UNIVERSITY OF CALIFORNIA, Oakland, CA (US)

(72) Inventors: Dianne K. NEWMAN, Pasadena, CA (US); Melanie A. SPERO, Pasadena, CA (US); Manuela MARTINS-GREEN, Riverside, CA (US); John D. COATES, Walnut Creek, CA (US)

(21) Appl. No.: 17/234,656

(22) Filed: Apr. 19, 2021

Related U.S. Application Data
(60) Provisional application No. 63/012,036, filed on Apr. 17, 2020.

(51) Int. Cl. A61K 33/00; A61K 31/43; A61K 31/424; A61K 31/7056; A61K 31/545; A61K 31/496; A61K 31/4164; A61K 31/7036; A61K 31/431; A61K 31/546; A61K 31/427; A61K 33/14; A61K 33/18; A61K 38/48; A61K 38/18; A61K 31/355; A61K 31/198; A61K 9/06; A61K 9/00; A61P 31/02; A61P 17/02

(52) U.S. Cl. CPC A61K 33/00; A61P 17/02; A61K 31/424; A61K 31/7056; A61K 31/545; A61K 31/496; A61K 31/4164; A61K 31/7036; A61K 31/431; A61K 31/546; A61K 31/427; A61K 33/14; A61K 33/18; A61K 38/4886; A61K 38/1841; A61K 38/1808; A61K 38/1866; A61K 38/1858; A61K 31/355; A61K 31/198; A61K 9/06; A61K 9/0014; A61P 31/02; A61K 31/43

(57) ABSTRACT

Wound healing matrices, compounds, compositions, methods and systems comprising one or more chlorates, one or more chlorites, one or more antibiotics, one or more other antimicrobials, and/or one or more wound healing agents.

### CROSS-REFERENCE TO RELATED APPLICATION

The present application claims priority to U.S. Provisional Application No. 63/012,036 entitled “Wound Prevention and/or Treatment and Related Compounds, Matrices, Compositions, Methods and Systems” filed on Apr. 17, 2020 with docket number P2493-USP, the content of which is incorporated by reference in its entirety. The present application may also be related to U.S. provisional application No. 62/571,009, entitled “New Therapeutic Strategy to Combat Diverse Chronic Infections” filed on Oct. 11, 2017 with docket number CIT 7310-P3, to U.S. application Ser. No. 16/157,885 entitled “Methods and Systems to interfere with viability of bacteria and related Antimicrobials and Compositions” filed on Oct. 11, 2018, with docket number P2286-US, and to PCT application PCT/US2018/055416 entitled “Methods and Systems to interfere with viability of bacteria and related Antimicrobials and Compositions” filed on Oct. 11, 2018, with docket number P2286-PCT, the content of each of which is also incorporated by reference in its entirety.

### STATEMENT OF GOVERNMENT GRANT

This invention was made with government support under Grant No. AI146987 awarded by the National Institute of Health. The government has certain rights in the invention.

### FIELD

The present disclosure relates to wound treatment and/or prevention and related compounds, matrices, compositions methods and systems.

### BACKGROUND

Wound healing has been the focus of research in medical applications for a long time.

Although, various methods, systems and compositions have been developed to promote wound healing developing compositions, methods and systems that can stimulate and facilitate the wound healing process remains challenging with particular reference to treatment and/or prevention of chronic wounds.

### SUMMARY

Provided herein are methods, systems and related matrices, compounds and composition that in several embodiments can be used to effectively inhibit bacteria biofilm formation and/or disrupt bacterial biofilm formed within a wound of in an individual and/or to promote healing of the wound in the individual.

In preferred embodiments methods, systems and related compounds and composition herein described are used in connection with treatment of chronic wound to effectively promote the related healing.

According to a first aspect, a matrix, in particular a biofilm treatment matrix is described comprising an effective amount of a biofilm treatment agent embedded in a delivery matrix, the biofilm treatment agent comprising a chlorate alone or in combination with one or more antibiotics and/or antimicrobials. In preferred embodiments, in the biofilm treatment matrix comprises the biofilm treatment agent in an effective amount to disrupt bacterial biofilm in a chronic wound of the individual. In some embodiments, the biofilm treatment agent does not include nitrate.

According to a second aspect, a method is described of treating a wound in an individual. the method comprises contacting the wound with a biofilm treatment matrix herein described for a time and under conditions to inhibit bacteria biofilm formation and/or disrupt bacterial biofilm in the wound. In some embodiments, the method can further comprise after the contacting, applying to the wound an effective amount of a wound healing agent for a time and under conditions to promote re-epithelization and granulation tissue formation of the wound. In preferred embodiments, the wound is a chronic wound of the individual. In preferred embodiments, contacting the wound with a biofilm treatment matrix can be preceded by treating the wound with a therapeutically effective amount of chlorite.

According to a third aspect, a method and a system are described of treating a wound in an individual.

The method comprises contacting the wound of the individual with an effective amount of a biofilm treatment agent comprising a chlorate alone or preferably in combination with an antibiotic, the contacting performed for a time and under conditions to inhibit formation of a bacteria biofilm and/or disrupt a bacteria biofilm in the wound. The method further comprises, after the contacting, applying to the wound an effective amount of a wound healing agent to promote re-epithelization and granulation tissue formation of the wound. In preferred embodiments, contacting the wound with a biofilm treatment agent can be preceded by treating the wound with a therapeutically effective amount of chlorite.

The system comprises a biofilm treating agent comprising a chlorate alone or preferably in combination with one or more antibiotics and/or antimicrobials, and a wound healing agent, and optionally also chlorite for sequential administration in the method to of treating and/or preventing a wound in an individual herein described.

According to a fourth aspect, a method and a system are described of treating a wound in an individual.

The method comprises applying to the wound a wound healing agent, in combination with a biofilm treatment agent comprising a chlorate alone or preferably in combination with an antibiotic. In the method the wound healing agent is in an effective amount to promote re-epithelization and granulation tissue formation of the wound, and the biofilm treatment agent in an effective amount to inhibit formation of a bacteria biofilm in the wound. In preferred embodiments, the applying is performed after contacting the wound with an effective amount of a biofilm treatment agent comprising a chlorate alone or in combination with an antibiotic for a time and under condition to disrupt a biofilm in the wound. In preferred embodiments, the applying further comprises applying therapeutically effective amount of chlorite.

In addition, or in the alternative in further preferred embodiments the wound is a chronic wound of the individual.

The system comprises a wound healing agent in an effective amount to promote re-epithelization and granulation tissue formation of the wound, and a biofilm treatment agent in an effective amount to inhibit formation of a bacteria biofilm in the wound, the biofilm treatment agent comprising a chlorate alone or preferably in combination with an antibiotic. The system can further comprise a biofilm treatment agent comprising a chlorate alone or preferably in combination with an antibiotic in an effective amount to disrupt a biofilm in the wound. The system can further comprise an effective amount of chlorite as will be understood by a skilled person upon reading of the present disclosure.

According to a fifth aspect, a wound healing composition is described, the wound healing composition comprising a wound healing agent in an effective amount to promote re-epithelization and granulation tissue formation of the wound, and a biofilm treatment agent in an effective amount to inhibit formation of a bacteria biofilm in the wound, the biofilm treatment agent comprising a chlorate alone or preferably in combination with an antibiotic.

According to a sixth aspect, a method and a system are described of preventing a wound of an individual from becoming a chronic wound.

The method comprises contacting the wound with a biofilm treatment agent comprising a chlorate alone or preferably in combination with an antibiotic, the contacting performed for a time and under condition to inhibit formation of a bacteria biofilm and/or disrupt a bacteria biofilm in the wound thus preventing the wound from becoming a chronic wound. In some embodiments the method can further comprise applying to the wound an effective amount of a wound healing agent to promote re-epithelization and granulation tissue formation of the wound. In preferred embodiments, contacting the wound with a biofilm treatment agent can be preceded by treating the wound with a therapeutically effective amount of chlorite.
The system comprises a chlorate an antibiotic and optionally a wound healing agent for sequential use in the method to prevent a chronic wound herein described.

In some embodiments, wound healing matrices, compounds, compositions, methods and systems herein described can be used to treat and/or prevent systemic infections and/or chronic infections, such as pulmonary infections and/or infections associated with implanted medical devices.

The matrices, compounds, compositions, methods and systems herein described can result in better improvement of the chronic wound by destroying the bacteria in the wound and dissolving the biofilm more effectively either simultaneously or prior to applying the wound healing agents. The systems, compositions, matrices, and methods herein described can address multiple biological characteristics associated with wound healing such as prolonged inflammation, poor angiogenesis, thus promoting wound healing and closure.

The matrices, compounds, compositions, methods and systems herein described can be used in connection with applications wherein wound healing is desired. For example, matrices, compounds, compositions, methods and systems herein described can be used to develop therapeutic approaches and tools to treat and/or prevent wounds, and in particular to treat and/or prevent chronic wound formation. Additional exemplary applications include uses of the matrices, compounds, compositions, methods and systems herein described in several fields including basic biology research, applied biology, bio-engineering, biological analysis, aetiology, medical research, medical therapeutics, with particular reference to clinical applications and in additional fields identifiable by a skilled person upon reading of the present disclosure.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the present disclosure and, together with the detailed description and examples sections, serve to explain the principles and implementations of the disclosure.

FIG. 1 shows pictures of Wound progression after treatment with ciprofloxacin or a combination of ciprofloxacin and chlorate according to an exemplary embodiment of the instant disclosure. Treatment began 15 days after injury and infection, when a chronic wound has formed.

FIG. 2A and FIG. 2B show diagrams illustrating the results of experiments performed on Pseudomonas aeruginosa wild type or Pseudomonas aeruginosa ΔnarGHJI subjected to 1 mM chlorate for 72 hours. The diagram of FIG. 2A shows the percentage survival of P. aeruginosa wild type (WT) versus percentage survival P. aeruginosa ΔnarGHJI (ΔnarGHJII). The diagram of FIG. 2B shows the percentage chlorate remaining P. aeruginosa wild type (WT) versus percentage survival P. aeruginosa ΔnarGHJI (ΔnarGHJII).

FIG. 3A and FIG. 3B show charts illustrating the results of experiments performed on Pseudomonas aeruginosa wild type or Pseudomonas aeruginosa ΔhflC subjected to 1 mM chlorate over the course of 48 hours. The diagram of FIG. 3A shows the percentage survival of P. aeruginosa wild type (black dots/line) versus percentage survival P. aeruginosa ΔhflC (light gray dots and line). The diagram of FIG. 3B shows the percentage chlorate remaining P. aeruginosa wild type (black dots/line) versus percentage survival P. aeruginosa ΔhflC (light gray dots and line).

FIG. 4A and FIG. 4B show diagrams reporting the results of experiments performed on Pseudomonas aeruginosa Wild type and/or Nar mutated treated with various antibiotics as indicated and 10 mM chlorate (patterned block) or no chlorate (solid block). In particular, FIG. 4A shows results of experiments performed on Pseudomonas aeruginosa wild type treated with no antibiotics, Ciprofloxacin: 5 ug/mL, Piperacillin: 320 ug/mL, Tobramycin: 40 ug/mL Aztreonam: 160 ug/mL Ceftazidime: 40 ug/mL as indicated and Chlorate 10 mM (patterned black blocks), or no Chlorate (solid black block). FIG. 4B shows results of experiments performed on P. aeruginosa wild type (black) and P. aeruginosa nar mutant (grey) treated with no antibiotics, Ciprofloxacin: 5 ug/mL, Piperacillin: 320 ug/mL, Tobramycin: 40 ug/mL Aztreonam: 160 ug/mL as indicated and Chlorate 10 mM (patterned blocks), or no Chlorate (solid block).

### DETAILED DESCRIPTION

Provided herein are methods, systems, and related compounds and composition suitable for treating and/or preventing wounds and particularly chronic wounds.

The term “wound” as used herein indicates the result of a disruption of normal anatomic structure and function of an individual ((1988 (November), 1998 (May), 2007 (February)) (Lazarus, Cooper et al. 1994). Accordingly, wounds in the sense of the disclosure encompass a wide range of a defects or breaks in a tissue and/or organs of an individual, resulting from physical, chemical and/or thermal damage, and/or as a result of the presence of an underlying medical or physiological condition” as will be understood by a skilled person (Boateng, Matthews et al. 2008)).

Exemplary wounds comprise abrasions and tears of a tissue of an organ of an individual (e.g. skin) which can be caused by blunt and/or frictional contact with hard surfaces, such as when the an organ is torn, cut, or punctured (an open wound), when the organ is contused (a closed wound), as well as when the organ lesioned and comprise a region in an organ or tissue having abnormal structural change, e.g. following damage through injury or disease. (Boateng, Matthews et al. 2008))

Exemplary wounds comprise ulcers, like decubitis ulcers_(bedsores or pressure sores) and leg ulcers (venous, ischaemic or of traumatic origin). (Gilliland, Nathwani et al. 1988)) (Armstrong and Ruckley 1997)) (Hareendran, Bradbury et al. 2005), abscesses such as lesions caused by foreign bodies at the time of an injury, or by infections and tumors (Boateng, Matthews et al. 2008).

In particular wounds comprise abnormal structures in the body of an individual caused by mechanical forces (such as knives and guns but also surgical treatment), thermal sources, chemical agents, radiation, electricity and/or other sources identifiable by a skilled person (Boateng, Matthews et al. 2008) (Naradzay and Alson 2005). Wounds also comprise abnormal anatomic structure and function of organs and/or tissues in an individual resulting from conditions such as autoimmune diseases or disorders, infections such as viral infections, cancer, as well as chronic diseases such as diabetes.

Exemplary wounds comprise superficial wounds (affecting only a surface epithelium of the organ, e.g. epidermal skin), partial thickness wounds (also affecting a connective tissues, of the organ such as skin's deep dermal layers) and full thickness wound (further affecting deeper tissues of the organ such as subcutaneous fat in addition to the epidermis and dermal layers) (Boateng, Matthews et al. 2008); (Bolton and Van Rijswijk 1991) (Krasner, Kennedy et al. 1993).)

Exemplary wounds also comprise lesions in eyes, ears, stomach intestine and additional portions of the gastrointestinal tract, and in additional tissue organ or body part, including lesions occurring in pulmonary infections such as cystic fibrosis and additional conditions, and in general to chronic infections such as the ones associated with implanted medical devices in lungs and additional tissues and organs of an individual.

Wounds in the sense of the present disclosure can be categorized based on the related characteristics in connection with the wound healing process in the individual.

The term “wound healing” as used herein indicates a biological process directed to growth and tissue regeneration in the individual (Boateng. Matthews et al. 2008) In particular, during the wound healing process cellular and extracellular components of the injured tissue or organ interact to restore the integrity of the organ or tissue in interdependent and overlapping stages will be understood by a skilled person (Strodtbeck 2001) (Russell 2000) (BA 1999) (GS 1999) (Rothe and Falanga 1989), and (Shakespeare 2001)).

In particular, a wound heling process in the sense of the disclosure comprises hemostasis, inflammation, migration, proliferation and maturation phases (GS 1999) (DM 1999)).

The term “hemostasis” in the sense of the disclosure indicates a stage of wound healing characterized by the presence of by exudate (blood without cells and platelets), exudate components such as clotting factors, coagulation of the exudate, formation of a fibrin network, and production of a clot in the wound causing bleeding to stop (Boateng, Matthews et al. 2008) (Martin 1997).

The term “inflammation” in the sense of the disclosure indicates a stage of wound healing process characterized by release of protein-rich exudate, vasodilation through release of histamine and serotonin, presence of phagocytes and engulf dead cells forming necrotic tissue in the wound, sloughy (yellowish colored mass), and platelets aggregate as will be understood by a skilled person (Boateng, Matthews et al. 2008)). The inflammatory phase occurs almost simultaneously with hemostasis, sometimes from within a few minutes of injury to 24 h and lasts for about 3 days as also understood by a skilled person. (Boateng, Matthews et al. 2008))

The term “migration” in the sense of the disclosure indicates a stage of wound healing process characterized by movement of epithelial cells and fibroblasts to the injured area, regeneration and growth of fibroblast and epithelial cells accompanied by epithelial thickening. (Boateng, Matthews et al. 2008))

The term “proliferation” in the sense of the disclosure indicates a stage of wound healing process characterized by formation of granulation tissue, collagen synthesis and in-growth of capillaries and lymphatic vessels into the wound, formation of blood vessels, fibroblast proliferation and collagen thickening blood vessels decrease and oedema recedes, as will be understood by a skilled person. (Boateng, Matthews et al. 2008)). The proliferative phase occurs almost simultaneously or just after the migration phase (Day 3 onwards) and basal cell proliferation, which lasts for between 2 and 3 days, and continues for up to 2 weeks by which time blood vessels decrease and oedema recedes as will also be understood by a skilled person. (Boateng, Matthews et al. 2008))

The term “maturation” or “remodeling” in the sense of the disclosure indicates a stage of wound healing process characterized by formation of cellular connective tissue and strengthening of the new epithelium which determines the nature of the final scar. (Boateng, Matthews et al. 2008)) Cellular granular tissue is changed to an acellular mass from several months up to about 2 years.

A description of appearance of wound in connection with the wound heling process can be found in Table 1 of (Boateng, Matthews et al. 2008)) enclosed, as Appendix III in U.S. provisional 63/012,036 incorporated herein by reference in its entirety.

Wounds in the sense of the disclosure can be categorized in connection with the related progression and repairs in the healing process, in acute wounds and chronic wounds.

“Acute wounds” in the sense of the disclosure are “tissue injuries that heal completely, with minimal scarring, within the expected time frame, usually 8-12 weeks” (Boateng, Matthews et al. 2008),) (see also (Percival 2002)).

Conversely, a “chronic wound” or a “complex wound” in the sense of the disclosure indicates wounds that fail to proceed through the normal phases of wound healing in an orderly and timely manner and often stall in the inflammation phase of healing. In particular, the wording “chronic wound” refers a wound subjected to a disruption of the orderly sequence of events during the wound healing process which slows down or prevent healing of the wound (Harding, Morris et al. 2002) (Moore, McCallion et al. 2006)).

Typically, a chronic wound is wound not healed in 4 weeks and in some cases over 4 weeks, beyond, 12 weeks or later (Harding, Morris et al. 2002)) typically following repeated tissue insults, underlying physiological conditions, pathological conditions (e.g. persistent infections) treatment of the individual and/or other patient related factors (Boateng, Matthews et al. 2008)). If healed, a chronic wound can often reoccur. (Moore, McCallion et al. 2006))

Typically a chronic wound is a characterized by a high level of oxidative stress compared with non-chronic wounds and with tissue and organs with no lesions, Oxidative stress (OS) is present in tissues and cells when there is an imbalance between the levels of reactive oxygen species (ROS) and the ability of antioxidants in the tissues and cells to remove these species and repair the damage they cause, as will be understood by a skilled person (see (Martins-Green 2020) enclosed as Appendix VI n U.S. provisional 63/012,036 incorporated herein by reference in its entirety.

Oxidative stress can be detected by detecting expression levels of enzymes that produce ROS, e.g. XCT or Slc7a11, which can have up to a 8.6 times fold increase, Nox4 which can have up to 2.1 or 3 fold increase and Hmox1 which can have up to 4.5 fold increase, in chronic wounds determined by Nanostring analysis during the first 48 hrs of chronicity initiation. Additional methods to detect oxidative stress comprise measuring the levels of DNA/RNA damage, lipid peroxidation, and protein oxidation/nitration, directed to measure reactive oxygen species indirectly, as well as additional methods identifiable by a skilled person.

Typically, a chronic would is also characterized by hypoxic or anoxic conditions. In particular, in a chronic wound the pO2 is typically halved compared to a non-chronic wound. For example, chronic wound surfaces on skin have been identified to be hypoxic at ˜37 mmHg, with a mean pHe of ˜6.8 even in absence of an epidermal barrier absent in most areas. Additionally, it has been shown that one day after wounding pHe is above 8 and pO2 is ˜60 mmHg, and that both parameters decrease during epidermal barrier restoration in physiological healing (Schreml, Meier et al. 2014).

Exemplary chronic wounds in the sense of the disclosure comprise wounds presenting an extensive loss of the integument (skin, hair, and associated glands), wounds presenting tissue death and/or signs of circulation impairment and, as well as wounds resulting from a pathology (Boateng, Matthews et al. 2008); (Ferreira, Tuma Júnior et al. 2006)).

Exemplary chronic wounds further comprise wounds presenting an excess exudate which typically is more corrosive as it includes a relatively higher levels of tissue destructive proteinase enzymes (Boateng, Matthews et al. 2008) (Chen, Rogers et al. 1992); (Hareendran, Bradbury et al. 2005). Accordingly, chronic wounds comprise oedema caused by inflammation, reduced mobility and venous or lymphatic insufficiency and additional wounds presenting an excess exudate as will be understood by a skilled person (Boateng, Matthews et al. 2008); (Armstrong and Ruckley 1997)).

Exemplary chronic wounds also comprise wounds including foreign bodies and possibly presenting granuloma or abscess formation, and wounds presenting keloid (raised) scars resulting from excess collagen production in the latter part of the wound healing process. (Boateng, Matthews et al. 2008); (Martin 1997)).

Exemplary chronic wounds also comprise wounds presenting a persistent infection (e.g. Fournier's gangrene), and in particular infection of one of more pathogenic bacteria such as Staphylococcus aureus, Pseudomonas aeruginosa, Streptococcus pyrogenes and some Proteus, Clostridium and a Coliform. Typically, chronic wounds presenting persistent infections are infected with P. aeruginosa and/or S. aureus which significantly reduce skin graft healing (Boateng, Matthews et al. 2008); (Gilliland, Nathwani et al. 1988)).

Exemplary chronic wounds also comprise wounds of individuals in poor nutritional status (e.g. protein, vitamin (e.g. vitamin C) and mineral deficiencies) and/or of old age (Hemilä and Douglas 1999) (Rojas and Phillips 1999).

Exemplary chronic wounds further comprise wounds of individuals with underlying conditions such as diabetes and anaemia (Boateng, Matthews et al. 2008) (Patel 2005).) and/or under treatment of drugs such as glucocorticoids or other steroids capable of suppressing the body's inflammatory responses and thereby impede the inflammatory stage of wound healing (Pierce, Mustoe et al. 1989); (Beck, DeGuzman et al. 1993) (Chedid, Hoyle et al. 1996)).

Exemplary chronic wounds include diabetic foot ulcers, venous leg ulcers, pressure ulcers, decubitus ulcers (bedsores or pressure sores) and legulcers (venous, ischaemic or of traumatic origin) and others identifiable to a person skilled in the art.

In some embodiments, herein described, treating and/or preventing of a wound can be performed by inhibiting bacteria biofilm formation and/or disrupting bacterial biofilm in the wound with a biofilm treatment matrix, compositions, methods and systems based on a chlorate used alone or preferably in combination with one or more antibiotics and/or antimicrobials and possibly further in combination with a wound healing agent.

In preferred embodiments, matrix, compositions, methods and systems based on a chlorate can further comprise an effective amount of chlorite, typically administered before administration of chlorate alone or in combination with one or more antibiotics and/or antimicrobials and/or with wound healing agent, typically to inhibit formation of a bacterial biofilm.

As used herein the term “biofilm” indicates an aggregate of microorganisms in which cells adhere to each other on a surface. These adherent cells are frequently embedded within a self-produced matrix of extracellular polymeric substance (EPS). Accordingly, a biofilm, comprise a multicellular aggregate, attached to a surface or embedded within mucus. Biofilms can form on living or non-living surfaces and can be prevalent in natural, industrial and hospital settings. The microbial cells growing in a biofilm are physiologically distinct from planktonic cells of the same organism, which, by contrast, are single cells that can float or swim in a liquid medium. Formation of a biofilm begins with the attachment of free-floating microorganisms to a surface. These first colonists adhere to the surface initially through weak, reversible adhesion via van der Waals forces. If the colonists are not immediately separated from the surface, they can anchor themselves more permanently using cell adhesion structures such as pili. When the biofilm growth is balanced with that of biofilm dispersion, the biofilm is considered “mature.” Methods to quantify and measure biofilms will be known to a skilled person and can include, for example, the COMSTAT method of (Heydorn, Nielsen et al. 2000).

The term “chlorate” refers to chemical compounds containing chlorate oxyanion having the formula C103.

As used herein, “chlorine oxyanion” refers to an anion consisting of one or more oxygen atoms covalently bonded to a chlorine atom. Exemplary chlorine oxyanions include hypochlorite ion ClO⁻, chlorite ion ClO₂⁻, chlorate ion ClO₃⁻, and ClO₄⁻. Chlorine oxyanions are typically comprised within a salt. In particular, a salt of chlorine oxyanion as used herein contains the oxyanion together with a cation as a counterion.

The cation can be a metal cation and in particular the metal ion can have a charge of +1, +2, +3 or +4. Exemplary +1 cation includes Li¹⁺, Na¹⁺, K¹⁺, Cs¹⁺, and Ag¹⁺. Exemplary +2 cation includes Mg²⁺, Ca²⁺, Sr²⁺, Ba²⁺, Ni²⁺, Cu²⁺, Pb²⁺, Fe²⁺ and Zn²⁺. Exemplary +3 cation includes Al³⁺, and Fe³⁺. Exemplary +4 cation includes Ti⁴⁺, Zr⁴⁺.

The cation can be an oxycation which, as used herein, refers to a cation consisting of one or more oxygen atoms covalently bonded to another atom. Exemplary oxycation includes nitronium ion, NO₂¹⁺, and vanadyl ion, VO²⁺.

Exemplary chlorates include potassium chlorate, sodium chlorate, magnesium chlorate, silver chlorate, or in solution as chloric acid. Chlorate can be produced commercially or in laboratory settings. For example, metal chlorates can be prepared by adding chlorine to hot metal hydroxide such as potassium hydroxide or sodium hydroxide as will be understood by a person skilled in the art. The industrial scale synthesis can start from aqueous chloride solution instead of chlorine gas. Chlorate can also be isolated and purified from natural sources as will be understood by a person skilled in the art.

In the embodiments herein described, chlorate can be provided in any one of the amounts from 0.001 mM to 200 mM.

In some of these embodiments, chlorate can be administered in an amount from 0.001 mM to 10 mM, 20 mM 30, mM, 50 mM, 100 mM, 150 mM.

In some of these embodiments, chlorate can be administered in an amount from 0.01 mM to 10 mM, 20 mM 30, mM, 50 mM, 100 mM, 150 mM, or to 200 mM.

In some of these embodiments, chlorate can be administered in an amount from 0.1 mM to 10 mM, 20 mM 30, mM, 50 mM, 100 mM, 150 mM, or to 200 mM.

In some of these embodiments, chlorate can be administered in an amount from 1 mM to 10 mM, 20 mM 30, mM, 50 mM, 100 mM, 150 mM, or to 200 mM.

In some of these embodiments, chlorate can be administered in an amount from 0.001 mM to 30 mM.

In some of these embodiments, chlorate can be administered in an amount from 0.001 mM to 10 mM.

In some of these embodiments, chlorate can be administered in an amount from 0.001 mM to 1 mM.

In some of these embodiments, chlorate can be administered in an amount from 0.001 mM to 1 mM possibly 0.001 to 0.01 mM, or 0.01 to 1 mM.

In some of these embodiments, chlorate can be administered in an amount from 0.1 mM to 10 mM, or in an amount from 1 mM to 20 mM.

A skilled person will be able to identify a concentration for a particular application in view of the specific medium and specific manner of administration upon review of the present disclosure.

In embodiments, herein described an effective amounts of chlorate is an amount effective on Nar-containing bacteria in target environments which are hypoxic or anoxic, and/or where nitrate is present at micromolar concentrations, such as in chronic wounds, chlorate can be administered in absence of nitrate, as will be understood by a skilled person upon reading of the present disclosure.

In embodiments herein described, bacteria in the sense of the disclosure comprise Nar-containing bacteria. “Nar-containing bacteria” refer to the types of bacteria containing a gene set encoding cytoplasmic nitrate reductase (“Nar”), thus capable of conducting Nar-mediated nitrate respiration.

The term “Nar” “nitrate reductase” refers to a group of membrane-bound protein complexes that reduce nitrate to nitrite. Nar is bound to the inner membrane and its active site is located in the cytoplasm. In its reaction, Nar transfers electrons from a membrane-associated reduced quinone to nitrate, thus producing nitrite. This energetically favorable reaction is coupled to proton translocation to generate a proton motive force, which can ultimately be used to power the cell (e.g. ATP synthesis) (Corkery 2000) Nar is capable of using nitrate as an electron acceptor to reduce nitrate to nitrite during anaerobic respiration. as an alternative to using oxygen as a terminal electron acceptor. The membrane-bound Nar complex is composed of three subunits: a) a catalytic a subunit, encoded by narG, containing a molybdopterin cofactor; b) a soluble β subunit, encoded by narH, containing four [4Fe-4S] centers; and c) the γ subunit, encoded by narI, containing two b-type hemes. In some embodiments, formation of the Nar complex further requires a chaperone-like component required for the maturation of the αβ complex encoded by narJ gene.

Accordingly, in some embodiments, Nar in the sense of the current disclosure is encoded by a narGHJI operon possessed by the Nar-containing bacteria. narG, H, I encode the α, β, and γ subunit respectively, while narJ encodes the chaperone-like component required for the maturation of the αβ complex. The transcription of narGHJI is typically activated under hypoxic or anoxic conditions and further stimulated by the presence of nitrate.

The term “operon” is a functioning unit of DNA containing a cluster of genes under the control of a single promoter as will be understood by a person of ordinary skill in the art. The term “gene” as used herein indicates a polynucleotide encoding for a protein that in some instances can take the form of a unit of genomic DNA within a bacteria, plant or other organism.

The term “polynucleotide” as used herein indicates an organic polymer composed of two or more monomers including nucleotides, nucleosides or analogs thereof. The term “nucleotide” refers to any of several compounds that consist of a ribose or deoxyribose sugar joined to a purine or pyrimidine base and to a phosphate group and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers respectively to a nucleotide or nucleoside in which one or more individual atoms have been replaced with a different atom or a with a different functional group. Accordingly, the term polynucleotide includes nucleic acids of any length, and in particular DNA RNA analogs and fragments thereof.

The term “protein” as used herein indicates a polypeptide with a particular secondary and tertiary structure that can interact with another molecule and in particular, with other biomolecules including other proteins, DNA, RNA, lipids, metabolites, hormones, chemokines, and/or small molecules. The term “polypeptide” as used herein indicates an organic linear, circular, or branched polymer composed of two or more amino acid monomers and/or analogs thereof. The term “polypeptide” includes amino acid polymers of any length including full-length proteins and peptides, as well as analogs and fragments thereof. A polypeptide of three or more amino acids is also called a protein oligomer, peptide, or oligopeptide. In particular, the terms “peptide” and “oligopeptide” usually indicate a polypeptide with less than 100 amino acid monomers. A protein “sequence” indicates the order of the amino acids that form the primary structure.

As used herein the term “amino acid”, “amino acid monomer”, or “amino acid residue” refers to organic compounds composed of amine and carboxylic acid functional groups, along with a side-chain specific to each amino acid. In particular, alpha- or α-amino acid refers to organic compounds composed of amine (—NH2) and carboxylic acid (—COOH), and a side-chain specific to each amino acid connected to an alpha carbon. Different amino acids have different side chains and have distinctive characteristics, such as charge, polarity, aromaticity, reduction potential, hydrophobicity, and pKa. Amino acids can be covalently linked to form a polymer through peptide bonds by reactions between the amine group of a first amino acid and the carboxylic acid group of a second amino acid. Amino acid in the sense of the disclosure refers to any of the twenty naturally occurring amino acids, non-natural amino acids, and includes both D an L optical isomers.

Identification of a Nar-containing bacterium can be performed by various techniques. In some embodiments, Nar-containing bacteria can be identified by performing a database search using narG gene or amino acid sequence from a characterized Nar as a query sequence or reference sequence. Bacteria containing a gene or protein sequence having protein having at least 80% query coverage and at least 50% sequence similarity with respect to the reference sequence are identified as Nar-containing bacteria.

As used herein, “query coverage” refers to the percentage of the query sequence that overlaps the identified sequence. The term “sequence similarity” refers to a quantitative measurement of the similarity between sequences of a polypeptide or a polynucleotide. In particular, “sequence similarity” makes reference to the nucleotide bases or protein residues in the two sequences that are the same when aligned for maximum correspondence over a specified comparison window. When percentage of a sequence similarity is used in reference to proteins, it is recognized that residue position which are not identical often differ by conservative amino acids substitutions, where amino acid residues are substituted with a functionally equivalent residue of the amino acid residues with similar physiochemical properties and therefore do not change the functional properties of the molecule. Accordingly, similarity between two sequences can be expressed as percent sequence identity and/or percent positive substitutions. Widely used similarity searching programs, like BLAST, PSI-BLAST (Altschul S F 1997), SSEARCH (Smith T F 1981) (WR 1991), FASTA (Pearson W R 1988) and the HMMER3 (Johnson L S 2010) programs produce accurate statistical estimates, ensuring protein sequences that share significant similarity also have similar structures.

A functionally equivalent residue of an amino acid used herein typically refers to other amino acid residues having physiochemical and stereochemical characteristics substantially similar to the original amino acid. The physiochemical characteristics include water solubility (hydrophobicity or hydrophilicity), dielectric and electrochemical properties, physiological pH, partial charge of side chains (positive, negative or neutral) and other properties identifiable to a person skilled in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine and lysine are considered as functionally equivalent residues to histidine.

The similarity between sequences is typically measured by a process that comprises the steps of aligning the two polypeptide or polynucleotide sequences (a subject sequence and a reference sequence) to form aligned sequences, then detecting the number of matched characters in the subject sequence with respect to the reference sequence, i.e. characters similar or identical between the two aligned sequences, and calculating the total number of matched characters divided by the total number of aligned characters in each polypeptide or polynucleotide sequence, including gaps. The similarity result is expressed as a percentage of similarity.

As used herein, “reference sequence” is a defined sequence used as a basis for sequence comparison. A reference sequence may be a subset or the entirety of a specified sequence; for example, as a segment of a full-length protein or protein fragment. A reference sequence can comprise, for example, a sequence identifiable in a database such as GenBank and UniProt and others identifiable to those skilled in the art.

As understood by those skilled in the art, determination of percentage of similarity between any two sequences can be accomplished using a mathematical algorithm. Non-limiting examples of such mathematical algorithms are the algorithm of Myers and Miller (Myers and Miller 1988), the local homology algorithm of Smith et al. (Smith and Waterman 1981); the homology alignment algorithm of Needleman and Wunsch (Needleman and Wunsch 1970); the search-for-similarity-method of Pearson and Lipman (Pearson and Lipman 1988); the algorithm of Karlin and Altschul (Karlin and Altschul 1990), modified as in Karlin and Altschul (Karlin and Altschul 1993). Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA (Pearson and Lipman 1988), and TFASTA in the Wisconsin Genetics Software Package, Version 8 (available from Genetics Computer Group (GCG), 575 Science Drive, Madison, Wis., USA). Alignments using these programs can be performed using the default parameters.

In some embodiments, identification of a Nar-containing bacterium can be performed by performing a database search using narG amino acid sequence from P. aeruginosa NarG having sequence:

(SEQ ID NO 1)
MSHLLDRLQFFKKKQGEFADGHGETSNESRAWEGAYRQRWQHDKIVRSTHG
VNCTGSCSWKIYVKNGLITWETQQTDYPRTRPDLPNHEPRGCPRGASYSWY
IYSANRLKYPKVRKPLLKLWREARAQHGDPVNAWASIVEDAAKAKSYKSQR
GLGGFVRSSWDEVTEIIAAANVYTAKTYGPDRVIGFSPIPAMSMVSYAAGA
RYLSLIGGVCLSFYDWYCDLPPASPQIWGEQTDVPESADWYNSSYIIAWGS
NVPQTRTPDAHFFTEVRYKGTKTVSITPDYSEVAKLTDLWLNPKQGTDAAL
GMAFGHVILKEFHLDRPSAYFVDYCRQYTDMPMLVLLEEHAGGAFKPTRYL
RAADLADNLGQDNNPEWKTIAYDERSGGLVSPTGAIGYRWGESGKWNIAEL
DGRSGDQTRLQLSLLDGPEHACEVAFPYFAGQEHPHFKGVANDEVLLRRVP
FREIVAADGKRLRVATVYDLQMANYSIDRGLGGDNVATSYEDADTPYTPAW
QERITGVPAARATQVAREFADSADKTRGKAMVIIGAAMNHWYHMDMNYRAV
INMLMMCGCIGQSGGGWAHYVGQEKLRPQTGWAPLAFGLDWSRPPRQMNGT
SFFYLHSSQWRHEKLSMHEVLSPLADASRFAEHALDYNIQAERLGWLPSAP
QLNRNPLRIAAEAEAAGLPVADYVVRELKSGGLRFASESPDDPQNFPRNMF
IWRSNLLGSSGKGHEYMLKYLLGAKNGVMNDDLGKAGGPRPTEVDWVDDGA
EGKLDLVTTLDFRMSSTCMYSDIVLPTATWYEKDDLNTSDMHPFIHPLSAA
TDPAWEAKSDWEIYKAIAKKFSAVAEGHLGVEQDLVTVPLLHDTPTELAQP
FGGDGHDWKKGECEPMPGRNLPTLHLVERDYPNVYRKFTSLGPLLDKLGNG
GKGIGWNTEKEVKLVGDLNHRVVESGVSQGRPRIDSAIDAAEVVLALAPET
NGQVAVKAWEALSKITGREHAHLALPKEDEKIRFRDIQVQPRKIISSPTWS
GLEDEHVSYNAGYTNVHELIPWRTITGRQQFYQDHPWMQAFGEGFVSYRPP
VNTRTTEKLLNRKPNGNPEITLNWITPHQKWGIHSTYSDNLLMLTLSRGGP
IIWLSEHDAAKAGIVDNDWVEVFNANGAATCRAVVSQRVKDGMVMMYHAQE
RIVNVPGSETTGTRGGHHNSVTRVVLKPTHMIGGYAQQAWGFNYYGTVGCN
RDEFVVVRKMSKVDWLDEPRHGGLGGDALPQPLPQDI

as a reference sequence to search for homologs in public databases such as GenBank, UniProt, EMBL, and others identifiable to a person skilled in the art, using tools such as BLASTp and additional tools identifiable by a skilled person. In those embodiments, Nar-containing bacteria can be identified as those containing a protein having at least 80% query coverage and at least 50% sequence similarity compared to SEQ ID NO:1.

In particular, bacteria containing a gene or protein sequence having protein having at least 80% query coverage and at least 50% sequence similarity with respect to the SEQ ID NO: 1 of P. aeruginosa.

In some embodiments, identification of a Nar-containing bacterium can be performed by isolating cell membrane fractions and performing membrane fraction assay for nitrate reduction by detecting nitrite concentration. In addition, identification of a Nar-containing bacterium can also be performed by constructing a bacterial culture supplemented with chlorate and detecting chlorite concentration after incubation. The procedure can further comprise testing whether the chlorate reduction is inhibited by other compounds such as azide, cyanide, and thiocyanate. (Moreno-Vivian, Cabello et al. 1999)

In some embodiments, identification of a Nar-containing bacterium can be performed by culture-independent techniques, such as performing whole genome sequencing and BLAST annotated protein sequences to a P. aeruginosa Nar as described herein. In particular, whole genome sequencing can be performed using culture-dependent methods (e.g. isolate bacterium, culture, extract DNA, sequence) or through culture-independent methods (e.g. single-cell sequencing. Another culture independent technique that can be performed to detect Nar-containing bacteria is sequencing a community's metagenome from an environment, with or without culturing. Metagenomic will provide an indication of whether Nar exists within a community, or with enough depth/coverage allow one to assemble genomes of individuals from the community.

In some embodiments, identifying nar-containing bacterium can be performed by detecting genes encoding Nar. For example, detecting genes encoding Nar can be performed by detecting sequences of one or more of the narG, narH, narJ and narI in the genome, transcriptome, or proteome of one or more candidate bacteria as described above. Exemplary techniques that can be used to detecting sequences of one or more genes (e.g. where the genome is known), comprises computer-based tools for comparing gene sequences, transcript sequences, or protein sequences, such as those using the Basic Local Alignment Search Tool (BLAST) or any other similar methods known to those of ordinary skill in the art.

In some embodiments, detecting genes encoding Nar in the one or more candidate Nar-containing bacteria can be performed by detecting the genes and/or related transcript in the one or more candidate bacteria. Exemplary techniques comprise wet bench approaches such as DNA sequencing, PCR, Southern blotting, DNA microarrays, or other methods of hybridization of DNA or RNA probes to DNA, wherein probes are attached to a label capable of emitting a signal such as radiolabeling, fluorescence, luminescence, mass spectroscopy or colorimetric methods. Exemplary probes that can be used comprise primers from known narG, narH, narJ and narI and/or related transcript as will be understood by a skilled person.

In some embodiments, detecting genes encoding Nar in the one or more candidate bacteria strains can be performed by detecting transcripts of narG, narH, narJ and narI. Exemplary techniques comprise RNA sequencing, PCR, quantitative PCR, Northern blotting, in situ hybridization, RNA microarrays, or other methods of hybridization of DNA or RNA probes to RNA.

In some embodiments, detecting genes encoding Nar in the one or more candidate bacteria strains can be performed by detecting proteins encoded by narG, narH, narJ and narI. Exemplary techniques comprise proteomics, antibody-based methods including immunohistochemistry, immunofluorescence, western blotting, or any other method of protein detection.

In embodiments herein described, the conditions and parameters to use probes/primers to detect narG, narH, narJ and narI can be varied to permit lower or higher threshold or stringency of detection, to ensure hybridization within at least 80% sequence identity at gene level in view of the specific primers/probes selected. For example, use of oligonucleotides comprising one or more degenerated nucleotide bases or using an antibody that binds to more highly conserved protein regions, can require modification of the detection conditions as will be understood by a skilled person.

In an exemplary embodiment, the detection can be done, for example, by isolating genomic DNA from a candidate strain and performing PCR using primer sequences designed to amplify narG gene from known Nar-containing bacteria, including the primers listed in the Example section. Alternatively, RNA samples can be isolated from the candidate and these transcripts can be sequenced, and expression of the narG gene can be detected by identification of this gene using homology-based computational identification (e.g. BLAST).

Other methods for identifying a bacterium capable of nitrate respiration would be identifiable to a skilled person upon reading of the present disclosure.

In some embodiments, exemplary Nar-containing bacteria include Pseudomonas aeruginosa, Staphylococcus aureus, Proteus spp. Escherichia coli, Propionibacterium acnes, Mycobacterium tuberculosis. Exemplary bacteria in the sense of the disclosure can also include Pseudomonas, Actinomyces israelii, Actinomyces gerencseriae, Brevibacterium, Brevibacterium linens, Coryneform Bacteria, Corynebacterium diphtheria, Nocardia, Bacillus anthracis, Bacillus cereus, Brucella melitensis, Brucella suis, Brucella abortus, Burkholderia cenocepacia, Burkholderia pseudomallei, Pantoea agglomerans, Pectobacterium atrosepticum, Propionibacterium propionicus, Pseudomonas fluorescens, Salmonella enterica, Shigella species, Staphylococcus epidermidis, Streptomyces anulatus, and related species that contains Nar to facilitate various physiological functions identifiable to a skilled person upon reading of the present disclosure.

In some embodiments, the Nar-containing bacteria comprise P. aeruginosa, S. aureus, E. coli, wherein the nar operon is expressed under hypoxic/anoxic conditions. In particular, in P. aeruginosa, the presence of nitrate is known to further increase transcription of narGHJI.

In some embodiments where P. aeruginosa is the microorganism, sequences for the genes of the nar operon comprises

P. aeruginosa narG (SEQ ID NO: 2):
ATGAGTCACCTGCTCGACCGCCTGCAGTTCTTCAAGAAGAAGCAGGGCGAATTCGCCGATGGCCACGGCGAGACCAGCAACGAG
AGCCGCGCCTGGGAAGGTGCCTACCGGCAGCGCTGGCAGCACGACAAGATCGTGCGCTCCACCCACGGGGTGAACTGCACCGGC
TCCTGCTCCTGGAAGATCTACGTGAAGAACGGCCTGATCACCTGGGAAACCCAGCAGACCGACTACCCGCGCACCCGTCCGGACC
TGCCCAACCACGAGCCGCGCGGCTGCCCGCGCGGGGCCAGCTATTCCTGGTACATCTACAGCGCCAACCGCCTGAAGTACCCGAA
GGTGCGCAAGCCGTTGCTCAAGCTCTGGCGCGAGGCGCGGGCGCAGCACGGCGACCCGGTGAACGCCTGGGCCAGCATCGTCGA
GGACGCCGCCAAGGCGAAGAGCTACAAGAGCCAGCGCGGCCTGGGCGGCTTCGTCCGTTCCAGCTGGGACGAGGTCACCGAGAT
CATCGCCGCGGCCAACGTCTACACCGCCAAGACCTACGGTCCGGACCGGGTGATCGGCTTCTCGCCGATCCCGGCCATGTCGATG
GTCAGCTACGCCGCCGGCGCCCGCTACCTGTCGCTGATCGGCGGGGTCTGCCTGAGCTTCTACGACTGGTACTGCGACCTGCCGCC
GGCCAGCCCGCAGATCTGGGGCGAGCAGACCGACGTGCCGGAGTCGGCCGACTGGTACAACTCCAGCTACATCATCGCCTGGGG
CTCCAACGTGCCGCAGACGCGGACCCCGGACGCGCACTTCTTCACCGAGGTGCGCTACAAGGGCACCAAGACCGTCTCCATCACC
CCGGACTATTCCGAGGTGGCCAAGCTCACCGACCTCTGGCTCAACCCCAAGCAGGGCACCGACGCCGCGCTGGGCATGGCCTTCG
GTCACGTGATCCTGAAGGAATTCCACCTCGACCGGCCGAGCGCCTACTTCGTCGACTACTGCCGCCAGTACACCGACATGCCGAT
GCTGGTGTTGCTGGAAGAACACGCCGGCGGCGCGTTCAAGCCGACCCGCTACCTGCGCGCCGCCGACCTGGCGGACAACCTCGGC
CAGGACAACAACCCCGAGTGGAAGACCATCGCCTACGACGAGCGCAGCGGCGGGCTGGTCTCGCCCACCGGCGCCATCGGCTAT
CGCTGGGGCGAGTCAGGCAAGTGGAACATCGCCGAGCTGGACGGCAGGAGCGGTGACCAGACGCGCCTGCAACTGTCGCTGCTC
GATGGCCCGGAACATGCCTGCGAGGTGGCCTTCCCGTATTTCGCCGGGCAGGAGCACCCGCACTTCAAGGGCGTCGCCAACGACG
AGGTACTGCTGCGCCGGGTGCCGTTCCGCGAGATCGTCGCGGCGGACGGCAAGCGCCTGCGGGTGGCCACCGTCTACGACCTGCA
GATGGCCAACTACAGCATCGACCGCGGCCTGGGCGGCGACAACGTGGCGACCTCCTACGAGGACGCCGACACGCCCTATACCCC
GGCCTGGCAGGAGCGCATCACCGGCGTTCCGGCGGCGCGCGCGACGCAGGTCGCCCGCGAGTTCGCCGACAGCGCCGACAAGAC
CCGCGGCAAGGCGATGGTGATCATCGGCGCGGCGATGAACCACTGGTACCACATGGACATGAACTACCGCGCGGTCATCAACAT
GCTGATGATGTGCGGCTGCATCGGCCAGAGCGGCGGCGGCTGGGCGCACTATGTCGGCCAGGAGAAGCTGCGCCCGCAGACCGG
CTGGGCGCCGCTGGCCTTCGGCCTGGACTGGAGCCGGCCGCCGCGGCAGATGAACGGCACCAGCTTCTTCTACCTGCACAGCTCG
CAATGGCGCCACGAGAAGCTGTCGATGCACGAGGTGCTGTCGCCGCTGGCCGACGCCAGCCGCTTCGCCGAACACGCCCTGGACT
ACAACATCCAGGCCGAACGCCTCGGCTGGCTGCCGTCGGCGCCGCAACTGAACCGCAACCCGCTGCGCATCGCCGCCGAGGCCG
AGGCCGCCGGCCTGCCGGTCGCCGACTACGTGGTGCGCGAACTGAAGAGCGGCGGCCTGCGCTTCGCCAGCGAATCGCCGGACG
ATCCGCAGAACTTCCCGCGCAACATGTTCATCTGGCGCTCCAACCTGCTGGGCTCCTCCGGCAAGGGCCACGAGTACATGCTCAA
GTACCTGCTCGGGGCGAAGAACGGGGTGATGAACGATGACCTCGGCAAGGCCGGCGGTCCGCGTCCCACCGAGGTCGACTGGGT
TGACGACGGTGCCGAGGGCAAGCTCGACCTGGTCACCACCCTGGACTTCCGCATGTCCTCCACCTGCATGTACTCGGACATCGTCC
TGCCGACCGCTACCTGGTACGAGAAGGACGACCTCAACACCTCCGACATGCACCCCTTCATCCATCCGCTGTCGGCGGCCACCGA
TCCGGCCTGGGAAGCCAAGAGCGACTGGGAGATCTACAAGGCCATCGCCAAGAAGTTCTCCGCCGTCGCCGAAGGCCACCTCGG
CGTGGAGCAGGACCTGGTCACGGTGCCGCTGCTGCACGACACCCCCACCGAGCTGGCGCAGCCGTTCGGCGGCGACGGCCATGA
CTGGAAGAAGGGCGAGTGCGAGCCGATGCCGGGACGCAACCTGCCGACGCTGCACCTGGTCGAGCGCGACTACCCGAACGTCTA
CCGCAAGTTCACCTCGCTCGGTCCGCTGCTGGACAAGCTGGGCAACGGCGGCAAGGGCATCGGCTGGAACACCGAGAAGGAAGT
GAAGCTGGTCGGCGACCTCAACCATCGCGTCGTCGAGAGCGGCGTCAGCCAGGGCCGCCCGCGCATCGACAGCGCCATCGACGC
CGCTGAGGTGGTCCTCGCCCTGGCTCCGGAAACCAACGGCCAGGTCGCGGTCAAGGCCTGGGAAGCGCTGTCGAAGATCACCGG
CCGCGAGCATGCCCACCTGGCGCTGCCCAAGGAAGACGAGAAGATCCGCTTCCGCGACATCCAGGTGCAGCCGCGCAAGATCAT
CTCCAGCCCGACCTGGTCCGGCCTCGAGGACGAGCACGTCAGCTACAACGCCGGCTACACCAACGTCCACGAGCTGATCCCGTGG
CGCACCATCACCGGTCGCCAGCAGTTCTACCAGGACCACCCGTGGATGCAGGCGTTCGGCGAAGGCTTCGTCAGCTACCGGCCGC
CGGTCAACACCCGGACCACCGAGAAACTGTTGAACAGGAAGCCCAACGGCAACCCGGAGATCACCCTGAACTGGATCACCCCGC
ACCAGAAATGGGGCATCCACTCCACCTACAGCGACAACCTGCTGATGCTCACCCTGTCGCGCGGCGGTCCGATCATCTGGCTCAG
CGAGCACGACGCGGCCAAGGCCGGGATCGTCGATAACGACTGGGTCGAGGTGTTCAACGCCAACGGCGCGGCGACCTGCCGCGC
GGTGGTCAGCCAGCGGGTCAAGGACGGCATGGTGATGATGTACCACGCCCAGGAACGCATCGTGAACGTACCCGGCAGCGAGAC
CACCGGCACCCGTGGCGGCCACCACAACTCGGTGACCCGCGTGGTGCTCAAGCCGACCCACATGATCGGCGGCTACGCCCAGCAG
GCCTGGGGCTTCAACTACTACGGCACGGTCGGCTGCAACCGCGACGAGTTCGTCGTGGTGCGCAAGATGAGCAAGGTCGACTGGC
TGGACGAACCCCGCCACGGCGGACTCGGCGGCGACGCCCTGCCGCAACCGCTGCCCCAGGACATTTGA
P. aeruginosa narH (SEQ ID NO: 3):
ATGAAAATTCGTTCGCAAGTCGGCATGGTGCTGAACCTCGACAAGTGCATTGGTTGCCACACCTGCTCGATCACCTGCAAGAACG
TCTGGACCAGCCGCGAAGGCATGGAGTACGCCTGGTTCAACAACGTCGAGACCAAGCCCGGGATCGGCTACCCGAAGGAATGGG
AAAACCAGGAGAAGTGGAAGGGCGGCTGGGTGCGCGCGGCGGACGGTTCGATCCGCCCGCGCATCGGCGGCAAGTTCCGCGTGC
TGGCGAACATCTTCGCCAACCCGGACCTGCCCGAGATCGACGACTACTACGAACCGTTCGACTTCGATTACCAGCACCTGCATAC
CGCGCCCAAGGCCGAGCACCAGCCGGTGGCGCGCCCGCGCTCGCTGGTCTCCGGGCAGCGCATGGAGAAGATCGAGTGGGGCCC
GAACTGGGAGGAGATCCTCGGCACCGAGTTCGCCAAGCGGCGCAAGGACAAGAACTTCGACCAGGTCCAGGCGGACATCTACGG
TGAGTACGAGAACACCTTCATGATGTACCTGCCGCGCCTCTGCGAGCACTGCCTGAACCCGGCGTGCGTGGCGTCCTGCCCGAGC
GGGGCGATCTACAAGCGCGAGGAGGACGGCATCGTCCTGATCGACCAGGACAAGTGCCGCGGCTGGCGGATGTGCATCTCCGGC
TGCCCGTACAAGAAGATCTACTTCAACTGGAAGAGCGGCAAGTCCGAGAAGTGCATCTTCTGCTACCCGCGCATCGAGGCCGGCC
AGCCCACTGTCTGCTCGGAGACCTGCGTCGGGCGCATCCGCTACCTCGGCGTGCTGCTCTACGACGCCGACCGCATCCACGAAGT
GGCCAGTTGCGAGAACGAGCGCGAGCTGTACGAGAAGCAACTGGAGATCTTCCTCGATCCGTTCGACCCGGCGGTGATCGCCCAG
GCGCGCAAGGACGGGGTGGCCGACAGCGTCATCGAGGCGGCGCAGAAGTCGCCGGTGTACAAGCTGGCGATGGACTGGAAGCTG
GCCCTGCCGCTGCACCCGGAATACCGCACGCTGCCGATGGTCTGGTACGTGCCACCGCTGTCGCCGATCCAGAACGCCGCCGCCG
AGGGGCACATCGGCAGCGACGGGGTGATCCCGGACGTGGAGTCGCTGCGCATCCCCGTGCAGTACCTGGCCAACCTGCTCACCGC
CGGCGACACCGCGCCGGTGCTGCTGGCGCTCAAGCGCCTGCTGGCGATGCGCGCCTACAAGCGCGCCGAGCACGTCGAAGGCCG
CCAGGACCTGGAGGTGCTGGCCAAGGTCGGGTTGAGCGTGGAGCAGGTGGAGGAGATGTACCGCTACCTGGCCATCGCCAACTA
CGAGGATCGCTTCGTGATCCCCAGCGCGCACCGCGAGGAAGCGCTTTCCGATGCCTTCGCCGAGCGTTCCGGCTGCGGCTTCAGC
TTCGGCAACGGCTGTTCCGGCGGCAGCAACTCCGCCGTCAACCTGTTCGGCGGCAAGCCGACCAACCGCCGCGACGTGATCCAGG
TCGTGCAGATCCAGGAGTGA
P. aeruginosa narJ (SEQ ID NO: 4):
ATGAACGATCACAGCCAACTGTTCCGCCTGCTCGCCCTGCTGCTCGACTATCCACGCGCCGAGCTGCGCGAGGAGAGCCTCGGCC
TGCATGCGCTGATCCGCACCTGCGAACTGCCGGAAGCGCTGCGCGACGGCCTCGCGGCGCTGCTCAACGAGCTCTGCCAGGGCGA
CCTGCTGGACGTCCAGGCGCGCTACGACGGTCTCTTCGAGCGCGGCCGCTCGGTCTCGCTGCTGCTCTTCGAGCACGTCCACGGCG
AGAGCCGCGACCGTGGCCAGGCGATGGTCGACCTGCTCGACCGCTATACCGGGGCCGGCCTGCAGATCGACGTACCGGAGCTGC-
CGGACTACCTGCCGCTGTACCTCGAATACCTGTCGCTGCTGCCGTTCGCGGCGGCCAGCGAAGGGCTCGCCGAAGTGGCGCACAT
CCTCGGCCTGCTGGCGCTGCGCCTGGAGGAACGCGGCAGCGCCTACGCGGCGATTTTCGAGGCGTTGCTGGAACTCGGCGGCGAG
CGCCCGGACCTCGGCGCGTTGCGTCGCGACCAGGCCCAGGAACAGCGCGACGACAGCCTGGAGGCCATCGACCGGGCCTGGGAG
GAAACCCCGGTGAGCTTCACCGACCCTGCCGGCGGTTGCCCGTCGAGCAGCGGCCGCCGTCCGACGGCGTCCACCGAACAACCAT
TGCAATGGGTCGCCCAGCCGGTACCGCAGATGCAGTACCGCGCGGCCCGCGAAGGAGTCTGA
P. aeruginosa narI (SEQ ID NO: 5):
ATGTCGACCAATCTTCTGTTCTTCGGGATCTATCCCTATGTCGCGCTGCTGATCTGCCTGGTCGGCAGCTGGGCGCGCTTCGACCT
CTCGCAGTACACCTGGAAGGCCGGTTCCAGCCAGATGCTCAGCAAGAAGGGCATGCGGGTATACAGCAACCTGTTCCACGTCGGCG
TGCTGTTCATCCTCGCCGGCCACTTCGTCGGCCTGCTGACCCCGGCCTCGGTCTACCACCACCTGATCAGCACCGAGAACAAGCAA
CTGCTGGCGATGGTCTCCGGCGGCTTCTTCGGCGTGCTCTGCTTCATCGGCCTGAGCGGACTGATCCTGCGCCGCCTGACCGACGC
GCGGGTGCGCGCCACCGGCAACGCCTCTGACCTGATGATCCTGCTGGTGCTCTACGCCCAGCTGATCCTCGGCCTCTCCACCATCG
TCGCCTCGACCCATCACATGGATGGCTCGGTGATGGTGATGCTCGCCGACTGGGCCCAGGCCATCGTCACCCTGCGTCCGCTGGC
GGCGGCCGAAGCCATCGCGCCGGTGGGCCTGGTCTACAAGCTGCACGTCGGCCTGGGCCTGACCCTGTTCGTGCTGTTCCCCTTCA
CCCGCCTGGTGCACATCGTCAGCGCGCCGGTGTGGTACCTGGGCCGGCGCTACCAGATCGTGCGTCAGAAACGTCCTGCCTGA

Other NarG sequences similar to P. aeruginosa NarG include NarG from gamma proteobacteria such as E. coli having 98% query coverage and 83% sequence similarity with respect to SEQ ID NO:1, NarG sequence from the gram positive bacterium S. aureus having 97% query coverage and 67% sequence similarity with respect to SEQ ID NO:1, and the NarG sequence from the delta proteobacterium Anaeromyxobacter sp. Fw109-5 having 95% query coverage and 61% sequence similarity with respect to SEQ ID NO:1.

In some embodiments, the Nar-containing bacteria can comprise additional genetic features, such as mutations and or other changes which typically affect the rate of nitrate respiration and that in some instances can occur over the course of the bacteria's infection.

For example, in some embodiments, the Nar-containing bacteria herein described can comprise a lasR mutation in which lasR function is defective or lost. lasR is a gene encoding a quorum-sensing regulator, so the loss of this gene has pleiotropic effects (D'Argenio, Wu et al. 2007). One phenotypic trait of lasR mutants is their decreased rates of oxygen respiration and increased rates of Nar-dependent nitrate respiration (Hoffman, Richardson et al. 2010). lasR mutants have been isolated from human infections such as bacteremia, pneumonia, chronic wounds, and CF (Smith, Buckley et al. 2006) and more resistant to some antibiotics. The prominence of lasR mutants has been documented in CF studies, where they are among the most frequently isolated mutants from CF patients (Smith, Buckley et al. 2006) and their presence is associated with worse lung function (Hoffman, Kulasekara et al. 2009). lasR mutants are also more resistant to antibiotics commonly used to treat P. aeruginosa infections (D'Argenio, Wu et al. 2007, Hoffman, Richardson et al. 2010).

In particular, in some embodiments, Nar-containing bacteria comprising a lasR mutation show increased rates of nitrate respiration and chlorate consumption and reduce chlorate more rapidly than the wild type bacteria does.

Accordingly, in some of the embodiments herein described, the methods, systems, compounds, and composition herein described are directed to interfere with viability of Nar-containing bacteria comprising a lasR mutation.

Detailed description on Nar and Nar-containing organisms can be found in copending application U.S. Ser. No. 16/157,885 filed on Oct. 11, 2018 published as US 2019/0142864, incorporated by reference in its entirety.

Exemplary Nar-containing bacteria that can be found in wounds and/or infections are reported in Table 1

TABLE 1

| Nar-containing bacterium | Disease |
|---|---|
| Actinomyces israelii | Actinomycosis |
| Actinomyces gerencseriae | |
| Propionibacterium propionicus | |
| Bacillus anthracis | Anthrax |
| Bacillus cereus | infection |
| Brucella melitensis | Brucellosis |
| B. suis | |
| B. abortus | |
| Corynebacterium diphtheriae | Diphtheria |
| Escherichia coli and other species | various infections |
| Burkholderia pseudomallei | Melioidosis |
| Streptococcus pneumoniae | Pneumococcal infection |
| Salmonella species | Salmonellosis |
| Shigella species | Shigellosis |
| Mycobacterium tuberculosis | Tuberculosis |
| Some Staphylococcus species, | various infections |
| including S. aureus and S. | including chronic |
| epidermidis | wounds |
| Pseudomonas aeruginosa | chronic wounds, |
| | ventilator-associated |
| | pneumonia, and Cystic |
| | fibrosis |

Additional exemplary Nar-containing bacteria comprise Burkholderia cepacia complex, Achromobacter xylosoxidans, Stenotrophomonas maltophilia and additional species.

In preferred embodiments, matrix, compositions, methods and systems based on a chlorate is administered combination with one or more antibiotics.

The term “antibiotics” as used herein refers to a type of antimicrobial used in the treatment and prevention of bacterial infection. Some antibiotics can either kill or inhibit the growth of bacteria. Others can be effective against fungi and protozoans. The term “antibiotics” can be used to refer to any substance used against microbes. Antibiotics are commonly classified based on their mechanism of action, chemical structure, or spectrum of activity. Most antibiotics target bacterial functions or growth processes. Antibiotics having bactericidal activities target the bacterial cell wall, such as penicillins and cephalosporins, or target the cell membrane, such as polymyxins, or interfere with essential bacterial enzymes, such as rifamycins, lipiarmycins, quinolones and sulfonamides. Antibiotics having bacteriostatic properties target protein synthesis, such as macrolides, lincosamides and tetracyclines. Antibiotics can be further categorized based on their target specificity. “Narrow-spectrum” antibacterial antibiotics target specific types of bacteria, such as Gram-negative or Gram-positive bacteria. “Broad-spectrum” antibiotics affect a wide range of bacteria.

In matrices, agents, compositions, methods and systems of the present disclosure antibiotics are comprised in a therapeutically effective amounts that can be identified by a skilled person based on the specific agent, wound and route of administration as will be understood by a skilled person.

In matrices, agents, compositions, methods and systems of the present disclosure the combined administration of a chlorate with one or more antibiotics is known and expected to result in a synergic antibacterial effect resulting from the combined administration as shown in Example 3 of the instant disclosure.

In embodiments herein described, suitable antibiotics that can be used in combination with chlorate include ampicillin, kanamycin, ofloxacin, Aminoglycosides, Carbapenems, Ceftazidime, Cefepime, Ceftobiprole, Fluoroquinolones, Piperacillin, Ticarcillin, tobramycin, aztreonam, coliston, tazobactam, and others (or combinations of these antibiotics) that can be readily recognized by a person skilled in the art.

Additional antibiotics that can be used in combination with one or more chlorates herein described include Amoxicillin and clavulanic acid (Augmentin®), Methicillin, oxacillin, nafcillin, cloxacillin, dicloxacillin, cabenicillin, ticarcillin, piperacillin, mezlocillin, azlocillin, ticarcillin and clavulanic acid (Timentin®), piperacillin and tazobactam (Zosyn®), cephalexin, cefdinir, cefprozil, cefaclor, cefuroxime, sulfisoxazole, erythromycin/sulfisoxazole, tobramycin, amikacin, gentamicin, erythromycin, clarithromycin, azithromycin, tetracycline, doxycycline, minocycline, tigecycline, ciprofloxacin, levofloxacin, vancomycin, linezolid, imipenem, meripenem, and aztreonam.

As a person of ordinary skill in the art would understand, the antibiotics herein listed can be selected for treating infections and/or reducing inflammation caused by bacteria including Staphylococcus (S. aureus and S. epidermidis), Pseudomonas (P. aeruginosa), Burkholderia cepacia, Escherichia coli, Enterococcus spp., Corynebacterium spp., and some mycobacteria. In some embodiments, antibiotics can be selected to treat infections and/or reduce inflammation caused by the bacteria listed in Table 2 below.

TABLE 2
Bacteria found in chronic wounds

| Bacteria | References |
|---|---|
| Acinetobacter sp. | (Gjødsbøl et al., 2006, 2012; Dowd et al., 2008a; James et al., |
| | 2008; Gontcharova, 2010; Wolcott et al., 2016) |
| Anaerococcus sp. | (Gardner et al., 2013; Smith et al., 2016; Wolcott et al., 2016) |
| Bacillus sp. | (Gjødsbøl et al., 2006; Dowd et al., 2008b, 2008a; |
| | Gontcharova, 2010; Wolcott et al., 2016) |
| Corynebacterium sp. | (Gontcharova, 2010; Gjødsbøl et al., 2012; Gardner et al., |
| | 2013; Scales and Huffnagle, 2013; Smith et al., 2016; Wolcott |
| | et al., 2016) |
| Enterobacter sp. | (Gjødsbøl et al., 2006; Dowd et al., 2008a; James et al., 2008; |
| | Smith et al., 2016; Wolcott et al., 2016) |
| Enterobacter cloacae | (Gjødsbøl et al., 2006, 2012) |
| Enterococcus sp. | (Dowd et al., 2008a; James et al., 2008; Scales and Huffnagle, |
| | 2013; Smith et al., 2016; Wolcott et al., 2016) |
| Enterococcus faecalis | (Gjødsbøl et al., 2006, 2012; Wolcott et al., 2016) |
| Escherichia sp. | (Dowd et al., 2008a; James et al., 2008; Gontcharova, 2010; |
| | Gjødsbøl et al., 2012; Scales and Huffnagle, 2013) |
| Escherichia coli | (Gjødsbøl et al., 2006; Dowd et al., 2008a) |
| Finegoldia sp. | (Gontcharova, 2010; Gardner et al., 2013; Wolcott et al., |
| | 2016) |
| Finegoldia magna | (Smith et al., 2016; Wolcott et al., 2016) |
| Paenibacillus sp. | (Dowd et al., 2008a) |
| Peptoniphilus sp. | (Dowd et al., 2008a; Gardner et al., 2013; Smith et al., 2016; |
| | Wolcott et al., 2016) |
| Porphyrornonas sp. | (Gardner et al., 2013) |
| Prevotella sp. | (Gontcharova, 2010; Gardner et al., 2013; Scales and |
| | Huffnagle, 2013; Smith et al., 2016; Wolcott et al., 2016) |
| Propionibacterium sp. | (Gontcharova, 2010; Wolcott et al., 2016) |
| Propionibacterium acnes | (Wolcott et al., 2016a) |
| Pseudomonas sp. | (Gjødsbøl et al., 2006; Dowd et al., 2008a; James et al., 2008; |
| | Gontcharova, 2010; Scales and Huffnagle, 2013; Smith et al., |
| | 2016; Wolcott et al., 2016) |
| Pseudomonas aeruginosa | (Gjødsbøl et al., 2006, 2012; Scales and Huffnagle, 2013; |
| | Wolcott et al., 2016) |
| Staphylococcus sp. | (Dowd et al., 2008a; James et al., 2008; Gontcharova, 2010; |
| | Gardner et al., 2013; Smith et al., 2016; Wolcott et al., 2016) |
| Staphylococcus epidermidis | (Scales and Huffnagle, 2013; Wolcott et al., 2016) |
| Streptococcus sp. | (Dowd et al., 2008a; James et al., 2008; Gontcharova, 2010; |
| | Scales and Huffnagle, 2013; Smith et al., 2016; Wolcott et al., |
| | 2016) |
| Turicibacter sp. | Wolcott et al., 2016b |

In some embodiments, suitable antibiotics comprise antibiotics effective against Pseudomonas aeruginosa such as Aminoglyco sides, Carbapenems, Ceftazidime, Cefepime, Ceftobiprole, Fluoroquinolones, Piperacillin, Ticarcillin, tobramycin, aztreonam, coliston, and others (alone or in combination) that can be recognized by a skilled person.

Exemplary antibiotics that can be used in combination with chlorate for treating chronic wounds include tobramycin, amoxicillin, clavulanic acid, clindamycin, aminoglyco sides, ciprofloxacin, cefalosporines, metronidazole and others identifiable to a person skilled in the art.

In preferred embodiments, antibiotics that can be used in combination with chlorate for treating chronic wounds include Ciprofloxacin:, Piperacillin, Ceftazidime, Aztreonam:, and Tobramycin: In some embodiments, one or more of Ciprofloxacin: 5 ug/mL, Piperacillin: 320 ug/mL, Ceftazidime: 40 ug/mL, Aztreonam: 160 ug/mL, and Tobramycin: 40 ug/mL can be administered alone or in combination.

In some embodiments, the effective amount of one or more antibiotics is a therapeutically effective amount which can be obtained according to drug description, FDA guidance, or recommendations by Centers for Disease Control and Prevention (CDC), Infectious Diseases Society of America (IDSA) or other health protection agencies as will be understood by a person skilled in the art.

For example, in some embodiments, the antibiotic can comprise amikacin at concentration from 2 to 64 μg/ml, and in particular 8 μg/ml, 16 μg/ml and 64 μg/ml, in particular to target Clinically significant aerobic gram-negative bacilli.

In some embodiments, the antibiotic can comprise ampicillin at a concentration of from 2 to 32 μg/ml, and in particular 4 μg/ml, 6 μg/ml and 32 μg/ml, in particular to target Clinically significant aerobic gram-negative bacilli.

In some embodiments, the antibiotic can comprise ampicillin/sulbactam from 2 to 32/16 μg/ml, and in particular 4/2 μg/ml, 16/8, g/ml and 32/168 μg/ml, in particular to target Clinically significant aerobic gram-negative bacilli.

In some embodiments, the antibiotic can comprise cefazolin at a concentration of from 4 to 64 μg/ml, and in particular 4 μg/ml, 16 μg/ml and 64 μg/ml, in particular to target Clinically significant aerobic gram-negative bacilli.

In some embodiments, the antibiotic can comprise cefepime at a concentration of from 1 to 64 μg/ml, and in particular 2 μg/ml, 8 μg/ml, 16 μg/ml and 32 μg/ml, in particular to target Clinically significant aerobic gram negative bacilli.

In some embodiments, the antibiotic can comprise cefoxitin at a concentration of from 4 to 64 μg/ml, and in particular 8 μg/ml, 16 μg/ml and 32 μg/ml, in particular to target Clinically significant aerobic gram-negative bacilli.

In some embodiments, the antibiotic can comprise ceftazidime at a concentration of from 1 to 64 μg/ml, and in particular at 1 μg/ml, 2 g/ml, 8 μg/ml and 32 μg/ml, in particular to target Clinically significant aerobic gram negative bacilli.

In some embodiments, the antibiotic can comprise ceftriaxone at a concentration of from 1 to 64 μg/ml, and in particular at 1 μg/ml, 2 μg/ml, 8 μg/ml and 32 μg/ml, in particular to target Clinically significant aerobic gram negative bacilli.

In some embodiments, the antibiotic can comprise Ciprofloxacin at a concentration of from 0.25 to 4 μg/ml, and in particular at 0.5 μg/ml, 2 μg/ml, and 4 μg/ml, in particular to target Clinically significant aerobic gram-negative bacilli.

In some embodiments, the antibiotic can comprise Gentamicin at a concentration of from 1 to 16 μg/ml, and in particular at 4 μg/ml, 16 μg/ml, and 32 μg/ml, in particular to target Clinically significant aerobic gram-negative bacilli.

In some embodiments, the antibiotic can comprise Levofloxacin at a concentration of from 0.12 to 8 μg/ml, and in particular at 0.25 μg/ml, 0.5 μg/ml, and 2.8 μg/ml, in particular to target E. cloacae, E. coli, K. pneumoniae, P. mirabilis, P. aeruginosa, S. marcescens, A. baumannii, A. lwoffli, C. koseri, C. freundii, E. aerogenes, E. sakazakii, K. oxytoca, M. morganii, P. agglomerans, P. vulgaris, Pv. rettgeri, Pv. stuartii, P. fluorescens, C. sakazakii.

In some embodiments, the antibiotic can comprise Meropenem at a concentration of from 0.25 to 16 μg/ml, and in particular at 0.5 μg/ml, 2 μg/ml, 6 μg/ml and 12 μg/ml, in particular to target E. coli, K. pneumoniae, P. aeruginosa, P. mirabilis, Acinetobacter spp., C. freundii, E. cloacae, K. oxytoca, M. morganii, P. vulgaris, S. marcescens, A hydrophila, C. diversus, H. alvei, P. multocida, Salmonella spp., Shigella spp.

In some embodiments, the antibiotic can comprise Nitrofurantoin at a concentration of from 16 to 512 μg/ml, and in particular at 16 μg/ml, 32 μg/ml, and 64 μg/ml, in particular to target Clinically significant aerobic gram-negative bacilli.

In some embodiments, the antibiotic can comprise Piperacillin/Tazobactam at a concentration of from 4/4 to 128/4 μg/ml, and in particular at 2/4 μg/ml, 8/4 g/ml, 24/4 μg/ml, 32/4, μg/ml, 32/8 μg/ml, and 48/8 μg/ml in particular to target Clinically significant aerobic gram negative bacilli A. baumannii, E. coli, K. pneumoniae, P. aeruginosa, C. koseri, M. morganii, P. mirabilis, P. vulgaris, Pv. rettgeri, Pv. stuartii, S. enterica.

In some embodiments, the antibiotic can comprise Tobramycin at a concentration of from 1 to 16 μg/ml, and in particular at 8 μg/ml, 16 μg/ml, and 64 μg/ml, in particular to target Clinically significant aerobic gram-negative bacilli.

In some embodiments, the antibiotic can comprise Trimethoprim/Sulfamethoxazole at a concentration of from 1 to 16 μg/ml, and in particular at 1/19 g/ml, 4/76 g/ml, and 16/304 μg/ml, in particular to target Klebsiella spp., Enterobacter spp., M. morganii, P. vulgaris, P. mirabilis, S. sonnei, S. flexneri, Eco(+ETEC)**, C. sakazakii.

In a further exemplary embodiment, of the biofilm treatment matrix, compositions, methods and systems herein described the antibiotic is Ciprofloxacin at 20 μg/100 μl and the chlorate is 100 μl of a 10 mM solution.

Additional therapeutic concentrations of antibiotics can be identified by a skilled person. according to drug description, FDA guidance, or recommendations by Centers for Disease Control and Prevention (CDC), Infectious Diseases Society of America (IDSA) or other health protection agencies as will be understood by a person skilled in the art.

In preferred embodiments, matrix, compositions, methods and systems based on a chlorate can further comprise an effective amount of chlorite, typically administered before administration of chlorate alone or in combination with one or more antibiotics, additional antimicrobial and/or wound healing agents.

The term “chlorite” refers to chemical compounds containing chlorite oxyanion having the formula ClO₂⁻. Chlorite refers to a salt of chlorous acid (HClO₂).

Exemplary chlorites include potassium chlorite, sodium chlorite, magnesium chlorite. Chlorite is the strongest oxidizer of the chlorine oxyanions on the basis of standard half-cell potentials under acid conditions.

In some embodiments, sodium chlorite is dissolved in water to make a sodium chlorite aqueous solution having a concentration of 0.1% to 25% by weight based on the total weight of the solution. Preferably the concentration of sodium chlorite in water is 0.5 to 5%. More preferably concentration of sodium chlorite in water is 1 to 3%.

In one embodiment, a composition of sodium chlorite solution in water having a concentration of 0.1% to 25% by weight based on the total weight of the solution is topically applied to a wound in a subject wherein the wound is covered by the composition. In some embodiment, the composition is applied topically to a wound every 8 hours, or every 24 hours, or until the wound is healed.

In some embodiments, a composition of sodium chlorite solution in water is administered orally to a subject in need of the medication, in an amount of 0.1 to 180 mg per day, preferably 0.5 to 50 mg per day, more preferably 1 to 10 mg per day.

In some embodiments, the sodium chlorite can be formulated as an ointment composition comprising sodium chlorite and paraffin, wool fat, beeswax, macrogols, emulsifying wax, cetrimide or vegetable oil (olive oil, arachis oil, coconut oil) or a combination there of in an amount of 0.1% to 25% by weight based on the total weight of the ointment composition, preferably 0.5 to 5% by weight based on the total weight of the ointment composition, more preferably 1 to 3% by weight based on the total weight of the ointment composition. In some embodiment, the ointment composition is applied topically to a wound every 8 hours, or every 24 hours, or until the wound is healed.

In some embodiments, the chlorite can administered systemically in an amount between 3 mg/kg b.w. to 32 mg/kg per day (Lubbers and Bianchine 1984) [cited in WHO, 2008 (Lubbers, Chauhan et al. 1981) [cited in WHO, 2008] (Lubbers, Chauan et al. 1982) [cited in WHO, 2008]. (Lubbers, Chauhan et al. 1984) [cited in WHO, 2008]. (Lubbers, Chauhan et al. 1984) [cited in WHO, 2008].

The term “antimicrobial” as used herein indicates a substance that kills or inhibits the growth of microorganisms such as bacteria, fungi, or protozoans. Antimicrobial either kills microbes (microbiocidal) or prevent the growth of microbes (microbiostatic).

Exemplary antimicrobial that can be used in combination with chlorate for treating chronic wounds include sterile saline or hydrogel, povidone-iodine solutions, cadexomer iodine, hypochlorous acid, collagenase and others identifiable to a person skilled in the art.

In some embodiments, compositions, methods and systems herein described can further comprise at least one wound healing agent.

The wording “wound healing agents” refer to agents that can stimulate and/or accelerate any one of the stages during the wound healing process, including inflammation, proliferation and remodeling as will be understood by a skilled person. In matrices, agents, compositions, methods and systems of the present disclosure wound healing agents are comprised in a therapeutically effective amounts that can be identified by a skilled person based on the specific agent, wound and route of administration as will be understood by a skilled person.

In some embodiments, wound healing agents comprise growth factors, which are substance capable of stimulating cell division, migration, differentiation, protein expression and enzyme production and/or cell proliferation, in an organ or tissue of the individual “Growth factor” at Dorland's Medical Dictionary 2011 (2011); (Mandla, Davenport Huyer et al. 2018)). In particular, the wound healing properties of growth factors are typically mediated through stimulation of angiogenesis and cellular proliferation, which affects both the production and the degradation of the extracellular matrix and also plays a rolein cell inflammation and fibroblast activity. (Komarcević 2000)) and affect the inflammatory, proliferation and migratory phases of wound healing. (Ten Dijke and Iwata 1989).)

Growth factors typically comprise secreted proteins or steroid hormones, signaling molecules between cells. Examples are cytokines and hormones that bind to specific receptors on the surface of their target cells and promote cell differentiation and maturation. Exemplary target cells re keratinocytes and fibroblasts which are involved in re-epithelialization and collagen deposition, respectively (Mandla, Davenport Huyer et al. 2018); (Mayet, Choonara et al. 2014)).)

Exemplary growth factors possibly comprised in wound healing compositions, and related biomimetic matrix, methods and systems comprise epidermal growth factor (EGF), platelet derived growth factor (PDGF), fibroblast growth factor (FGF), transforming growth factor (TGF-b1), insulin-like growth factor (IGF-1), human growth hormone and granulocyte-macro-phage colony-stimulating factor (GM-CSF)(Boateng, Matthews et al. 2008); (Steenfos 1994) (Greenhalgh 1996).)

Preferred growth factors comprise GM-CSF with particular reference to in full thickness wounds (Mann, Niekisch et al. 2006)). epidermal growth factor (EGF), (Mandla, Davenport Huyer et al. 2018)) with particular reference to combined treatment with silversulphadiazine (Lee, Leem et al. 2005)), PDGF with particular reference to treatment where granulation tissue and reepithelialization is desired (such as) in human patients with diabetic foot ulcers. (Mandla, Davenport Huyer et al. 2018); (Fang and Galiano 2008), (Miller 1999) (Pierce, Berg et al. 1991), (Greenhalgh, Sprugel et al. 1990), (Judith, Nithya et al. 2010), (Smiell, Wierman et al. 1999), (Gökşen, Balabanli et al. 2017). fibroblast growth factor (FGF), (Mandla, Davenport Huyer et al. 2018); (Greenhalgh, Sprugel et al 1990), (Choi, Ryu et al. 2016), (Choi, Lee et al. 2018), (Garcia-Orue, Gainza et al. 2017), (Lin, Lee et al. 2015)) and vascular endothelial growth factor (VEGF). (Mandla, Davenport Huyer et al. 2018); (Bao, Kodra et al. 2009), (Lord, Ellis et al. 2017), (Elgin, Dixit et al. 2001), (Long, Johnson et al. 2017) (Gu, Nguyen et al. 2004)).

A summary of growth factor modified materials and their corresponding strategies for growth factor encapsulation and delivery is reported in Table 1 of (Mandla, Davenport Huyer et al. 2018) enclosed as Appendix I n U.S. provisional 63/012,036 incorporated herein by reference in its entirety.

Therapeutically effective amount of growth factors can be identified by a skilled person in view of the specific factor and related formulation and route of administration as will be understood by a skilled person. For example. rhPDGF can be administered in an effective amount of 0.001% composition in the FDA approved Regranex. Additional, amounts can be identified by a skilled person.

In some embodiments, wound healing agents comprise supplements such as vitamins and mineral supplements (Boateng, Matthews et al. 2008) (Wallace 1994). including vitamins A, C, E as well as zinc and copper. (Boateng, Matthews et al. 2008) comprised in an effective amount identifiable by a skilled person in view of the specific supplement as well as timing formulation and route of administration.

In some embodiments, supplements included in wound healing compositions, biomimetic matrix, and related compositions methods and systems herein described, comprise Vitamin A, in particular in embodiments where treatment is directed to promote epithelial cell differentiation, (Boateng, Matthews et al. 2008) (Linder 1991)) collagen synthesis and bone tissue development. (Flanigan 1997, Boateng, Matthews et al. 2008)) normal physiological wound healing as well as reversing the corticosteroid induced inhibition of cutaneous wound healing and post-operative immune depression. (Ehrlich, Tarver et al. 1973, Boateng, Matthews et al. 2008).

In some embodiments, supplements included in wound healing compositions, biomimetic matrix, and related compositions methods and systems herein described, comprise Vitamin C in particular in embodiments where treatment is directed to promote synthesis of collagen and other organic components of the intracellular matrix of tissues such as bones, skin and other connective tissues. (Boateng, Matthews et al. 2008) (Linder 1991)) normal responses to physiological stressors such as in accident and surgical trauma and the need for ascorbic acid increases during times of injury (Boateng, Matthews et al. 2008) (Pugliese P T. 1998.). immune function particularly during infection. (Boateng, Matthews et al. 2008) (Eaglstein, Davis et al. 1988)). (Martins-Green and Saeed, 2020) (Martins-Green 2020) (Taylor, Rimmer et al. 1974), (ter Riet, Kessels et al. 1995), (Fitzmaurice, Sivanani et al. 2011)).

In some embodiments, supplements included in wound healing combinations, compositions, biomimetic matrix, and related compositions methods and systems herein described, comprise Vitamin E in particular in embodiments where treatment is directed to promote wound healing. (da Rocha, Lucio et al. 2002)) preservation of important morphological and functional features of biological membranes. (Komarcević 2000). (Baumann and Md 1999)) antioxidant and anti-inflammatory activity (H 1995)) as well as promoting angiogenesis and reduces scarring (Kirschmann G 1996)). (Martins-Green and Saeed 2020) (Martins-Green 2020) (Rasik and Shukla 2000).

In some embodiments, supplements included in wound healing compositions, biomimetic matrix, and related compositions methods and systems herein described, comprise Zinc in particular in embodiments where treatment is directed to promote healing of leg ulcers through enhancement of reepithelialization (Ågren 1990)) upregulation of metallothioneins (Kietzmann and Braun 2006)), rapid healing of wounds retarded by corticosteroid treatment (Lansdown 2002). treatment of deep second-degree burn wounds, preferably in combination with FGF and EGF (Yang, Chai et al. 2001)) decrease of Staphylococcus load in the wound (Ågren, Ostenfeld et al. 2006)).

In some embodiments the wound healing agent is an antioxidant agent, which, as used herein indicates a compound that inhibits oxidation. In particular, in a biological environment, antioxidants inactivate reactive oxygen species (herein also ROS) by donating their electrons to these species and preventing them from capturing electrons from other important molecules such as DNA, proteins and lipids, thus protecting the environment against excessive oxidative stress (herein also OS) as will be understood by a skilled person. (Martins-Green and Saeed, 2020) (Martins-Green 2020).

In preferred embodiments, of the wound healing combination compositions biomimetic matrix and related compositions, methods and systems applied to chronic wounds, comprise at least one antioxidant. In some embodiments, the at least one antioxidant comprises an antioxidant operating through enzymatic and/or an antioxidant operating through non-enzymatic reactions that can occur intracellularly in the cytosol and/or in organelles such as the mitochondria or in the extracellular environment. (Martins-Green and Saeed, 2020) (Martins-Green 2020). Antioxidants are comprised in wound healing combination compositions biomimetic matrix and related compositions, methods and systems in a therapeutically effective amounts identifiable by a skilled person based on the specific antioxidant as well as formulation, method and route of administration.

Exemplary types of antioxidants, those that perform enzymatic reactions and those that are non-enzymatic in their effects are shown in Table I of (Martins-Green and Saeed 2002) (Martins-Green 2020) enclosed Appendix VI n U.S. provisional 63/012,036 incorporated herein by reference in its entirety, inclusive these antioxidants targeting reactions occurring in the extracellular microenvironment, others occur intracellularly in the cytosol and/or in organelles such as the mitochondria (Marczin, El-Habashi et al. 2003)).

In some embodiments, antioxidants included in wound healing combinations, compositions, biomimetic matrix, and related compositions methods and systems herein described, comprise one or more of superoxide dismutase (SOD), glutathione S-transferases (GSTs), glutathione peroxidases (GPx), NADP(H), catalase, heme-oxygenase 1 (HO-1), peroxiredoxins (Prdx), thioredoxin-1 (Trx-1) and -2 (Trx-2). (Martins-Green and Saeed, 2020 (Martins-Green 2020)).

In particular, in some embodiments, antioxidants included in wound healing combinations, compositions, biomimetic matrix, and related compositions methods and systems herein described, comprise Hemet-oxygenase 1 (HO-1) in particular in embodiments where treatment is directed to promote degradation of heme into CO and/or iron in the presence of O₂ and NADPH giving rise to biliverdin that is converted into bilirubin (Tenhunen, Marver et al. 1968) (Barañano, Rao et al. 2002), wound closure and angiogenesis resulting in increased wound healing. (Tenhunen, Marver et al. 1968) (Barañano, Rao et al. 2002), (Martins-Green and Saeed, 2020) (Martins-Green 2020).

In particular, in some embodiments, antioxidants included in wound healing combinations, compositions, biomimetic matrix, and related compositions methods and systems herein described, comprise peroxiredoxins and thioredoxins in particular in embodiments where treatment is directed to promote reduction of oxidative stress (Lu and Holmgren 2014) (Hanschmann, Godoy et al. 2013) reduction of H₂O₂ as well as a broadrange of peroxides (Hofmann, Hecht et al. 2002) (Wood, Schröder et al. 2003) detoxification of tissues and cells from peroxynitrite (Peshenko and Shichi 2001) (Kümin, Huber et al. 2006) rapid wound (Kümin, Huber et al. 2006) (Schäfer and Werner 2008), reduction of other proteins by cysteine thiol-disulfide exchange and reduction of inflammation (Yoshida, Nakamura et al. 2005). (Martins-Green and Saeed, 2020).

In particular, in some embodiments, antioxidants included in wound healing combinations, compositions, biomimetic matrix, and related compositions methods and systems herein described, comprise non-enzymatic antioxidants such as vitamin C (ascorbic), vitamin E (α-tocopherol), Vitamin D, glutathione, N-acetyl cysteine (NAC), alpha lipoic acid (aLA), carotenoids (e.g. lycopenes), bilirubin and uric acid. (Cano Sanchez, Lancel et al. 2018). (Vervaart and Knight 1996)] (Shukla, Rasik et al. 1997). (Kümin, Huber et al. 2006)

In some embodiments, antioxidants included in wound healing combinations, compositions, biomimetic matrix, and related compositions methods and systems herein described, comprise glutathione in particular in embodiments where treatment is directed to promote strength of the wound tissue. (Adamson, Schwarz et al. 1996) healing of wounds in diabetic individual (Mudge, Harris et al. 2002). Preferably in combination with Vit E(α-tocopherol) (Musalmah, Nizrana et al. 2005) (Wlaschek and Scharffetter-Kochanek 2005) (Loo, Wong et al. 2012) (Taylor and James 2005). (Martins-Green and Saeed, 2020) (Martins-Green 2020).

In some embodiments, antioxidants included in wound healing combinations, compositions, biomimetic matrix, and related compositions methods and systems herein described, comprise Vitamin D in particular in embodiments where treatment is directed to promote cancer prevention and inhibition of inflammation (Wiseman 1993), proliferation and migration of endothelial cells (Grandjean, Berthet et al. 2000).

In some embodiments, antioxidants included in wound healing combinations, compositions, biomimetic matrix, and related compositions methods and systems herein described, comprise Alpha-Lipoic Acid (α-LA) in particular in embodiments where treatment is directed to promote chelation of toxic heavy metal ions including Fe²⁺ and Cu²⁺. Fe²⁺ can react with H₂O₂ to produce Fe³⁺+OH⁻+OH⁻ (Fenton reaction) which can cause protein modification, lipid peroxidation and DNA damage, scavenging of OS (Petersen Shay, Moreau et al. 2008) regeneration of Vit E, Vit C, coenzyme Q10 and glutathione. (Martins-Green and Saeed, 2020).

In some embodiments, antioxidants included in wound healing combinations, compositions, biomimetic matrix, and related compositions methods and systems herein described, comprise N-acetyl-cysteine (NAC): in particular in embodiments where treatment is directed to promote antimicrobial activity in connection with biofilm formation and/or disruption, in particular in wounds infected by Pseudomonas aeruginosa, Escherichia coli, Staphylococcus epidermidis, Slreptococcus pneumnoniae, Staphylococcus aureus and Klebsiella pneumoniae (Kundukad, Schussman et al. 2017), (Marchese, Bozzolasco et al. 2003) (Gomes, Leite et al. 2012) (Mohsen et al 2015) (Khalaf, Kamal et al. 2015). N-acetyl-cysteine (NAC): can also be comprised in in particular in embodiments where treatment is directed to promote modulation of granulocyte function, increase IL-12 secretion, activation NF-κB pathway, decrease of metalloproteinase-9, IL-8, IL-6, and/or inflammatory cytokines and oxidative stress at normal levels (Cotgreave, Moldéus et al. 1991) (Hart, Terenghi et al 2004) (Raftos, Whillier et al. 2007) (Bernard, Lucht et al. 1984) (Aihara, Dobashi et al. 2000) (Paterson, Galley et al. 2003), burn wound healing (Yang. Zhu et al. 2013). Healing of incisional wound of diabetic and non-diabetic individual (Tsai, Huang et al. 2014) faster healing (Sen and Roy 2008) (Wlaschek and Scharffetter-Kochanek 2005) (Loo, Wong et al. 2012) (Taylor and James 2005) (Martins-Green and Saeed 2020).

In some embodiments, antioxidants included in wound healing combinations, compositions, biomimetic matrix, and related compositions methods and systems herein described, comprise other small molecules such as carotenoids (in particular lycopenes), bilirubin, and/or uric acid. (Martins-Green and Saeed 2020).

In some embodiments, antioxidants included in wound healing combinations, compositions, biomimetic matrix, and related compositions methods and systems herein described, comprise bilirubin in particular in embodiments where treatment is directed to promote healing, increased neovascularization and improved collagen deposition of diabetic wound (Ram, Singh et al 2014) and reduction of oxidative stress in wound tissues (Ram, Singh et al. 2016) (Martins-Green and Saeed 2020.

In some embodiments, antioxidants included in wound healing combinations, compositions, biomimetic matrix, and related compositions methods and systems herein described, comprise 6,8 dithio-uric acid in particular in embodiments where treatment is directed to promote wound healing protection of cells and in particular neural cells endothelial cells keratinocyte and fibroblasts from oxidative damage (Chigurupati, Mughal et al. 2010) (Nery, Kahlow et al. 2015) (Fitzmaurice, Sivamani et al. 2011). (Martins-Green and Saeed 2020).

In some embodiments, antioxidants included in wound healing combinations, compositions, biomimetic matrix, and related compositions methods and systems herein described, comprise herbal extracts such as curcumin and honey. (Martins-Green and Saeed 2020.)

In some embodiments, antioxidants included in wound healing combinations, compositions, biomimetic matrix, and related compositions methods and systems herein described, comprise curcunin in particular in embodiments where treatment is directed to promote increase in collagen content and wound contraction (Jagetia and Rajanikant 2004) and/or in treatment of excision wounds. (Panchatcharam, Miriyala et al. 2006) (Mohanty and Sahoo 2017) (Hussain, Thu et al. 2017). (Calabrese, Bates et al. 2008). (Martins-Green and Saeed 2020).)

In some embodiments, antioxidants included in wound healing combinations, compositions, biomimetic matrix, and related compositions methods and systems herein described, comprise honey in particular in embodiments where treatment is directed to promote, antimicrobial treatment (Alvarez-Suarez, Gasparrini et al. 2014) anti-inflammatory treatment. (Khan, Naz et al. 2017) early improvement in wound healing process. (Lindberg, Andersson et al. 2015) (Mukai, Komatsu et al. 2016). immunomodulatory treatment. (Niaz, Maqbool et al. 2017) (Jill, Cullum et al. 2015). (Martins-Green and Saeed 2020).

In some embodiments, antioxidants included in wound healing combinations, compositions, biomimetic matrix, and related compositions methods and systems herein described, comprise Factor-E2-related factor (Nrf2) in particular in embodiments where treatment is directed to improve healing under oxidative stress conditions in impaired wounds (Long, De La Vega et al. 2016) in particular in diabetic wounds (Zheng, Whitman et al. 2011) (Suzuki and Yamamoto 2017)]. (Long, De La Vega et al. 2016) (Uruno, Yagishita et al. 2015) (Jiang, Tian et al. 2014). (Martins-Green and Saeed 2020).

In some embodiments the wound healing agent is an anti-oxidant agent, such as N-acetyl cysteine, coenzyme Q (ubiquinol), vitamin A, vitamin C, vitamin E, glutathione, lipoic acid, carotenes, flavenoids, phenolics, and ergothioneine, melatonin, ellagic acid, punicic acid, luteolin, catalase, superoxide dismutase, peroxiredoxins, cysteine, or a physiological salt thereof, or a combination thereof. In some embodiments, the wound healing agent can be a free radical scavenger, a lipid peroxidation inhibitor, or a combination thereof.

Exemplary effective amounts of antioxidant agents comprise 0.1-3.0% NAC, 0.3% bilirubin ointment as well as 10 mg/kg of curcumin to increase collagen and 40 mg/kg for excision wounds (daily application).

Additional antioxidant agents, related concentration and applications are described in (Martins-Green and Saeed 20201 (Martins-Green 2020) enclosed, as Appendix VI in U.S. provisional 63/012,036 incorporated herein by reference in its entirety.

In some embodiments, wound healing compositions, combinations, and biometric matrix, as well as related compositions, methods and systems, comprise in addition to chlorate, at least one antioxidant, small molecules such as alpha-tocopherol (Vitamin E), n-acetyl cysteine (NAC), proteins such as cytokines, growth factors (e.g. EGF, VEGF, TGF beta, PDGF), and/or other bioactive molecules identifiable to a skilled person. In preferred embodiments at least one antibiotic is further comprised.

Additional wound healing agents and various approaches to apply for targeted wound therapy can be found in (Mandla, Davenport Huyer et al. 2018)) enclosed, as Appendix I in U.S. provisional 63/012,036 incorporated herein by reference in its entirety.

In some embodiments of the biofilm treatment matrix, compositions, methods and systems, herein described, chlorate can be administered together with one or more antibiotics either sequentially (such as the chlorate first and then the antibiotics) or in a single administration for a time period until the biofilm is disrupted. The wound can then be treated with wound healing compounds.

In embodiments of methods herein described, the method of treating and/or preventing chronic wound in an individual comprises contacting the chronic wound of the individual with the composition herein described comprising an effective amount of chlorate alone or in combination with an effective amount of antibiotics and/or antimicrobial. The contacting of the composition is performed for a time and under conditions to reduce antibiotic resistance and/or bacterial survivability, by producing chlorite which is toxic for the cell within the cytoplasm of the cell via Nar-mediated reduction of the chlorate into toxic chlorite. Accordingly, in embodiments herein described the contacting results in inhibition of viability of the Nar-containing bacteria via cytoplasmic chlorite production while minimizing the interference with the viability of possible neighboring cells lacking Nar.

The term “individual” as used herein in the context of treatment and/or prevention includes a single biological organism, including but not limited to, animals and in particular higher animals and in particular vertebrates such as mammals and in particular human beings.

Timing and dosages of administration of chlorate alone or in combination with one or more antibiotics and/or antimicrobials to treat and/or prevent bacterial infection herein described can vary depending on the individual treated, the effect to be achieved (treatment and/or prevention) and the severity of the infection as will be understood by a skilled person.

Suitable dosages can be used which provide the individual with a therapeutically effective amount or a prophylactically effective amount in accordance with the related embodiments of the disclosure. In particular, the term “effective amount” of one or more active ingredients refers to a nontoxic but sufficient amount of one or more drugs to provide the desired effect. For example, an “effective amount” of chlorate associated with the treating and/or preventing (herein also “therapeutically effective amount” or “pharmaceutically effective amount”) a condition in the individual in which bacterial infections are present, refers to a non-toxic but sufficient amount of the chlorate to provide the treatment and/or prevention of such condition in the individual. As another example, an “effective amount” of at least one antibiotic and/or antimicrobial associated with the treating and/or preventing bacterial infection in the individual refers to a non-toxic but sufficient amount of the at least one antibiotic and/or at least one antimicrobial to provide the treatment and/or prevention of the bacterial infection in the individual. A non-toxic amount for chlorate can be identified by a person skilled in the art based on the guidelines and health reference levels provided by health organizations such as WHO and environmental protection agencies such EPA.

In certain embodiments, administering the wound healing combinations and/or compositions of the disclosure can be performed by systemic administration. In some of those embodiments the systemic administration is performed by parenteral administration and more particularly intravenous, intradermic, and intramuscular administration. In some of those embodiments, systemic administration is performed by non-parenteral administration and more particularly intranasal, intratracheal, vaginal, oral, and sublingual administration.

Exemplary compositions for parenteral administration include but are not limited to sterile aqueous solutions, injectable solutions or suspensions including chlorate alone or in combination with antibiotics, antimicrobials and additional wound healing agents s will be understood by a skilled person.

In certain embodiments, administering the wound healing combinations and/or compositions of the disclosure can be performed by topical administration (Chu, Xiong et al. 2006) (Patrick, Rivey et al. 2006)) Topical administration include but is not limited to epicutaneous administration, inhalational administration (e.g. in asthma medications), enema, eye drops (E.G. onto the conjunctiva), ear drops, intranasal route (e.g. decongestant nasal sprays), and vaginal administration.

In some embodiments, the wound healing combinations and/or compositions of the disclosure can be administered transdermally using tools such as micro/nanocarriers that can pass through the skin barrier and stratum corneum or microneedles that can poke through the barrier and deliver the composition to the viable tissue underneath.

In some embodiments in which the wound healing is performed to treat and/or prevent systemic infections and/or chronic infections (e.g. pulmonary infections, and/or infections associated with the use of implanted medical devices) administration through intravenously, intramuscularly, or inhaled as an aerosol or via a nebulizer allows an effective delivery of the agents and compositions of the instant disclosure.

Accordingly, in some embodiments, a composition for treating and/or preventing chronic wound is described. The composition comprises one or more chlorate alone or in combination with one or more antibiotics, one or more antimicrobial, and/or one or more wound healing agents.

In some embodiments, a composition for treating and/or preventing chronic wound is described can be formulated as liquid (solutions, suspensions and emulsions) and semi-solid (ointments and creams) In particular, solutions such as are most effective in the initial stages of wound healing for reducing bacterial load and as debriding and desloughing agents to prevent maceration of healthy tissue by the removal of necrotic tissue from the fresh wound. Antimicrobial agents such as silver, povidone-iodine. (Misra and Nanchahal 2003) (Misra and Nanchahal 2003) and polyhexamethylene biguanide (Motta, Milne et al. 2004)) are sometimes incorporated into dressings to control or prevent infection. Physiological saline solution is usedfor wound cleansing to remove dead tissue and also washing away dissolved polymer dressings remaining in a wound. (Fukunaga, Naritaka et al. 2006); and (Dealey 1993).) Saline solution is alsoused to irrigate dry wounds during dressing change to aid removal with little or no pain. The major problem with liquid dosage forms, however, is short residence times on the wound site, especially where there is a measurable degree of suppuration (exuding) of wound fluid.

The composition herein described can further comprise one or more vehicles as would be identified by a skilled person.

The term “vehicle” as used herein indicates any of various media acting usually as solvents, carriers, binders or diluents for chlorate alone or in combination with antibiotics and/or additional wound healing agents, comprised in the composition as an active ingredient.

In some embodiments, where the composition is to be administered to an individual the composition can be a pharmaceutical wound healing composition and comprises chlorate alone or in combination with antibiotics and/or additional wound healing agents and a pharmaceutically acceptable vehicle.

In some embodiments, chlorate alone or in combination with antibiotics and/or additional wound healing agents can be included in pharmaceutical compositions together with an excipient or diluent. In particular, in some embodiments, pharmaceutical compositions are disclosed which contain chlorate alone or in combination with antibiotics and/or additional wound healing agents, in combination with one or more compatible and pharmaceutically acceptable vehicle, and in particular with pharmaceutically acceptable diluents or excipients.

The term “excipient” as used herein indicates an inactive substance used as a carrier for the active ingredients of a medication. Suitable excipients for the pharmaceutical compositions herein disclosed include any substance that enhances the ability of the body of an individual to absorb chlorate alone or in combination with antibiotics and/or additional wound healing agents. Suitable excipients also include any substance that can be used to bulk up formulations with chlorate alone or in combination with antibiotics and/or additional wound healing agents to allow for convenient and accurate dosage. In addition to their use in the single-dosage quantity, excipients can be used in the manufacturing process to aid in the handling of chlorate alone or in combination with antibiotics and/or additional wound healing agents. Depending on the route of administration, and form of medication, different excipients may be used. Exemplary excipients include but are not limited to antiadherents, binders, coatings disintegrants, fillers, flavors (such as sweeteners) and colors, glidants, lubricants, preservatives, sorbents.

The term “diluent” as used herein indicates a diluting agent which is issued to dilute or carry an active ingredient of a composition. Suitable diluents include any substance that can decrease the viscosity of a medicinal preparation.

In some embodiments, the composition can be in a form of a solution, patch, lotion, hydrogel, cream or embedded in a delivery matrix as will be understood by a skilled person.

In some embodiments of the compositions methods and systems herein described, chlorate alone or in combination with one or more antibiotics and the wound healing agents are administered to the wound in a single formulation which can be re-applied regularly.

In some embodiments, chlorate alone or in combination with one or more antibiotics and the wound healing agents can be comprised in a topical liquid or semi-solid formulations such as silver sulpha-diazine cream (Hudspith and Rayatt 2004)) and silver nitrate ointment (Liao, Huan et al. 2006)).

In some embodiments, the wound healing combination and/or composition herein described can be in the form of a lotion, hydrogel, solution (in water or PBS) or cream and can thus be delivered topically, e.g. directly into the wounds of an individual and in particular a patient. Alternatively, the composition herein described can be provided to an individual intravenously, intramuscularly, or inhaled as an aerosol or via a nebulizer.

In preferred embodiments, the wound healing combination and/or composition can be administered in a wound dressing configured to deliver the chlorate to the wound site. preferably further configured to cover the wound area and maintains a suitable condition supporting the healing process.

In preferred embodiments, the wound healing combination and/or composition is administered within dressings such hydrocolloid, alginate, collage (Queen, Orsted et al. 2004) ointment, film, foam, gel (Falabella 2006) (GM 2007)) in particular in primary or island dressings. (Van Rijswijk 2006) which can be used as debridement, antibacterial, occlusive, absorbent or adherence dressings (Purner S K 2000)).

As a person skilled in the art will understand, the wound dressing used herein in treating a wound are configured to cover the wound, preserve the body water content, be oxygen permeable to allow oxygen access to growing tissue, and prevent the growth of environmental pathogens without interfering with the wound healing. The utilized materials are configured to be immunocompatible, non-degradable, and should not support cell ingrowth and cellular adhesion so to avoid complications during their removal. The wound dressings used herein can preserve the activity of the composition components and should be able to release the components at the desired rate.

In some embodiments, wound dressings used herein are effective in removing wound exudates without dehydrating the tissues. The optimal material should guarantee gas and fluid permeability in order to absorb odors, maintain moist conditions and avoid dehydration and exudates accumulation which can result in the formation of necrotic tissue. Materials for wound dressings vary in terms of the origin of materials, physical forms, architecture, and properties.

Exemplary wound dressings are in the form of gauze, thin film, foam, hydrogels, hydrocolloids, membranes and other identifiable to a person skilled in the wound treatment. Detailed information about various materials used for wound dressing can be found in published literatures such as (Saghazadeh, Rinoldi et al. 2018) enclosed as Appendix II in U.S. provisional 63/012,036 incorporated herein by reference in its entirety.

In preferred embodiments, the wound healing combination and/or composition can be administered on a scaffolding material configured to deliver active agents to the wound site and preferably further hosting the endogenous cells and facilitate their growth and wound closure.

The term “scaffold” or “scaffolding material: as used herein indicates a structure comprised of a polymeric central component which is configured to deliver cells, drugs, and genes into the body.

A scaffolding material used herein encompass material configured to facilitate the tissue regeneration, restore the tissue function, and promote a rapid healing process preventing chronic wounds. Preferably, scaffolding material herein described are configured to have a degradation rate that matches the rate of tissue growth. Scaffolding material in the sense of the disclosure are configured to minimize immunogenicity and toxicity of the material and related byproducts of the degradation process.

Exemplary scaffolding materials include bioactive materials such as collagen, hyaluronic acid, chitosan or electrospun nanofibers that mimic the natural collagen fibers in ECM, synthetic polymers including polyurethanes and polyesters, hydrogel scaffolds, foams and spongy biomaterials, composite scaffolds, bi-layered scaffolds and others identifiable to a person skilled in the art. Detailed information about various materials used for scaffolding materials can be found in published literatures such as (Saghazadeh, Rinoldi et al. 2018) enclosed as Appendix II in U.S. provisional 63/012,036 incorporated herein by reference in its entirety.

In preferred embodiments, a scaffolding material used in wound healing combination and/or biomimetic matrix as well as in related compositions, methods and systems of the disclosure, is configured to adhere properly to the surrounding tissues and to have mechanical properties matching the mechanical properties of the native tissue or organ where the wound is located, to avoid the detachment and breakage over the course of healing. The scaffolding material used in wound healing combination and/or biomimetic matrix as well as in related compositions, methods and systems in the sense of the disclosure are preferably configured to control and in particular maintain its water content or are used in connection with administering approach devised to prevent material dehydration. (Saghazadeh, Rinoldi et al. 2018) (Wawrzyńska and Kubies 2018) and (Murray, West et al. 2019)).

In some embodiments, scaffolding material in the sense of the disclosure are configured to have a limited swelling capacity and maintain their shape over time. In these embodiments scaffolding materials can also be used as a depot of growth factors and the drug that are directly being delivered to the healing tissue. In this frame, engineered skin substitutes have been explored in order to create a 3-dimensional (3D) architecture that can mimic the ECM and reproduce the natural cell microenvironment. (Saghazadeh, Rinoldi et al. 2018) (Wawrzyńska and Kubies 2018) and (Murray, West et al. 2019)).

A most preferred scaffolding material for wound healing combination and/or biomimetic matrix as well as in related compositions, methods and systems of the present disclosure, should guarantee gas and fluid permeability in order to absorb odors, maintain moist conditions and avoid dehydration and exudates accumulation. (Saghazadeh, Rinoldi et al. 2018) (Wawrzyńska and Kubies 2018) and (Murray, West et al. 2019).

In some embodiments, the chlorate and wound healing agents alone or together with one or more antibiotics and/or one or more antimicrobials can be delivered in a delivery matrix. The delivery matrix can be designed to incorporate the components with high loading efficiency, controlled release while maintaining the bioactivity. (Saghazadeh, Rinoldi et al. 2018) (Wawrzyńska and Kubies 2018) and (Murray, West et al. 2019)).

In some embodiments, the composition herein described is embedded in a delivery matrix such as collagen (a natural component of tissues), hyaluronan (a natural component of tissues), hydrogels made of Poly(vinyl alcohol) (PVA), collagen-chitosan hydrogels, alginate matrices, carbopol gels, hydrocolloidal dressing, foam dressings, matrix enabling slow releases and/or differential releases, and others identifiable to a person skilled in the art. (Saghazadeh, Rinoldi et al. 2018) (Wawrzyńska and Kubies 2018) and (Murray, West et al. 2019).

Additional scaffolding materials and related features for use as delivery matrices in wound healing combination and/or biomimetic matrix as well as in related compositions, methods and systems of the present disclosure can be found for example in (Saghazadeh, Rinoldi et al. 2018), (Boateng, Matthews et al. 2008) (Wawrzyńska and Kubies 2018) and (Murray, West et al. 2019) enclosed as Appendix V, enclosed as Appendix II, Appendix III, Appendix IV, and Appendix V respectively in U.S. provisional 63/012,036 are herein incorporated herein by reference in their entirety.

Accordingly, in some embodiments, a biofilm treatment matrix for treating and/or preventing chronic wound is described, wherein a biofilm treatment agent comprising one or more chlorates alone or in combination with one or more antibiotics and/or antimicrobials is embedded in a delivery matrix.

The systems, compositions, and biofilm treatment matrices herein described can be applied to an individual in various stages of severity of biofilm development. In some embodiments, the systems, compositions, and biofilm treatment matrices herein described can be given to an individual in the early stages of wound healing (i.e. before a wound is defined as chronic) to inhibit the growth of Nar-containing bacteria in the wound or given to an individual after a wound is determined to be chronic to reduce viability of Nar-containing organisms.

In some embodiments, the systems, compositions, and biofilm treatment matrices can be administered shortly after stimulation of wound chronicity. This can be applied to bed sores and pressure sores which can be detected in very early stages.

In some embodiments, the composition for treating and/or preventing a chronic wound in an individual comprises chlorate in an effective amount between 0.001 mM and 200 mM and one or more antibiotics. In some embodiments, the chlorate is in an amount between 1 mM to 200 mM.

In some embodiments, the one or more antibiotics comprise tobramycin in an effective amount between 1 mg/kg/day and 10 mg/kg/day.

In some embodiments, the composition can comprise chlorate 5 mM-20 mM in 50-100 μl/application/day, antibiotic 5 μg/ml-20 μg/ml in 50-100 μl/application, and Wound Agents can comprise 100-500 mg/kg of NAC and 20 mg/kg-100 mg/kg of alpha-tocopherol.

In some embodiments, the composition can be administered once a day, twice a day, three times a day four times a day, or more often as necessary.

In some embodiments, the chlorate alone or in combination with one or more antibiotics and/or antimicrobials in the composition can be administered concurrently, combined in a single dosage form. For example, chlorate alone or in combination with one or more antibiotics and/or antimicrobials can be in a single vehicle dissolved in water or PBS.

In some embodiments, the chlorate alone or in combination with one or more antibiotics and/or antimicrobials can be administered at the same or at different times in separate dosage forms wherein antibiotic or antimicrobial can be administered before or after chlorate.

In some embodiments, methods herein described chlorate is administered in combination with an antibiotic to individuals in which the antibiotic treatment failed when isolate show in vitro sensitivity to the administered antibiotic. In those embodiments, the chlorate targets oxidant-starved pathogen populations, such as those found in chronic wound which are not reached by the antibiotic thus resulting in antibiotic tolerance and treatment failure.

In some embodiments, to stimulate wound healing once the biofilm of the wound is destroyed, a wound healing agent can be further applied to stimulate the healing of the wound.

Accordingly, the method can further comprise, following contacting the chronic wound of the individual with the composition herein describe, applying a wound healing agent in an effective amount to the chronic wound to stimulate the healing.

Exemplary wound healing agent comprise small molecules such as alpha-tocopherol (Vitamin E) (e.g. and 50 mg vitamin E per kg mouse or corresponding dosages in other individuals), n-acetyl cysteine (NAC) (e.g. 200 mg NAC per kg mouse or corresponding dosages in other individuals), proteins such as cytokines, growth factors (e.g. EGF, VEGF, TGF beta, PDGF), and other bioactive molecules identifiable to a skilled person. Additional wound healing agents and various approaches to apply for targeted wound therapy can be found in (Mandla, Davenport Huyer et al. 2018) and (Dhall, Do et al. 2014) enclosed as Appendix I, and Appendix VII, respectively in U.S. provisional 63/012,036 and which are incorporated herein by reference in their entirety.

The wound healing agents can be delivered in various delivery ways such as in protein itself, the cDNA of the proteins, cytokine and growth factor plasma rich fraction alone or embedded in a matrix.

In some embodiments of the methods herein described, the methods are provided to prevent wound chronicity and/or early stage of biofilm development. In these embodiments, contacting the chronic wound with an effective amount of the composition herein described can be performed shortly after wound development within hours or days. Examples of chronic wounds include bed sores and pressure sores that can be detected in very early stages, or diabetic foot ulcers as soon as a wounding event is recognized.

In some embodiments of the methods herein described, the methods are provided to treat chronic wounds and/or biofilm infections. In these embodiments, contacting the chronic wound with an effective amount of the composition herein described can be performed for wounds that are well-developed and have become infection. The composition can be embedded in matrices that permit slow releases such as over days or months or in matrices that allow for differential release.

The methods and composition herein described can allow for re-epithelialization, granulation tissue formation including angiogenesis and then remodeling of the tissue be stimulated.

As described herein, chlorate, nitrate, antibiotics, bacteria, antimicrobial agents and/or compositions herein described can be provided as a part of systems to perform any methods, including any of the assays described herein. The systems can be provided in the form of arrays or kits of parts.

In a kit of parts, the chlorate, antibiotics, antimicrobials, wound healing agent, and/or other reagents to perform the methods herein described can be included in the kit alone or in the combination with of one or more antibiotic and/or antimicrobial agents compositions. In particular in kit of parts for the treatment of an individual the chlorate, bacteria, and/or other reagents can be comprised together with the antibiotic and/or antimicrobial formulated for administration to the individual as well as additional components identifiable by a skilled person upon reading of the present disclosure.

### EXAMPLES

The matrices, compositions, compounds, methods and systems herein described are further illustrated in the following examples, which are provided by way of illustration and are not intended to be limiting.

A person skilled in the art will appreciate the applicability and the necessary modifications to adapt the features described in detail in the present section, to additional agents and related compositions, methods and systems according to embodiments of the present disclosure.

### Example 1: Chronic Wound Treatment

Due to the severity of the wound chronicity caused by PA14, in these experiments Pseudomonas aeruginosa was used that was isolated from a previous chronic wound in a mouse model. Skin was cleaned with iodine and ethanol and inoculated with 10⁴ CFUs of P. aeruginosa before a wound was made. This mimics what happens in humans in which it is the bacteria in the skin that invade the wound and create chronicity under the appropriate high oxidate stress levels. (Dhall, Do et al. 2014)).

Wounds were treated to stimulate high OS levels at wounding (see (Dhall, Do et al. 2014) for procedure), covered with Tegaderm and left alone. After wound chronicity was allowed to develop for 15 days with biofilm production, the Tegaderm was removed the wound debrided to mimic what happen in the clinician's office when the patient presents the case. The wound was then covered with a new Tegaderm film and treatment started immediately.

Two groups of animals were treated. Group #1 was treated with either ciprofloxacin every other day and Group #2 was treated with a combination of ciprofloxacin and chlorate on alternate days. Ciprofloxacin was used at 20 μg/100 μl and for chlorate 100 μl of a 10 mM solution was applied to the wound. Treatment in both groups decreased bacterial bioburden in the wounds and dismantled existing biofilm formed on the wounds. The combination treatment of both ciprofloxacin and chlorate led to more effective break down of the biofilm. While the treatments address the bacterial infections and lead to dismantling of the biofilm, the wounds remain open.

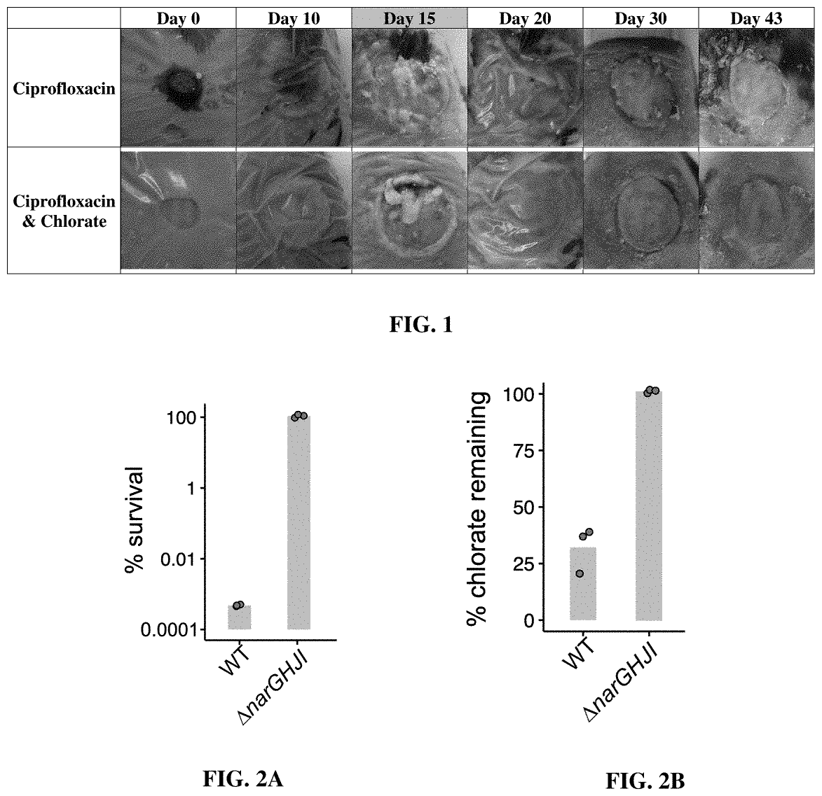

FIG. 1 illustrates an exemplary wound progression after treatment with ciprofloxacin or a combination of ciprofloxacin and chlorate. Treatment began 15 days after injury and infection, when a chronic wound has formed. From a comparison of the brighter/whiter top image of FIG. 1, showing the wound at the beginning of the treatment with the darker bottom image of FIG. 1 showing the wound at day 43, it is evident a significant reduction of the biofilm presence in the wound following the combined treatment with ciprofloxacin and chlorate as will be understood by a skilled person.

Accordingly, from the exemplary data shown in FIG. 1, it appears that the combination of the two treatments results in better improvement of the wound by dissolving the biofilm more effectively. This showing of destroying the bacteria and dismantling the biofilm is a major step in resolving chronicity as will be understood by a skilled person.

### Example 2: Chlorate Effects on Nar-Containing Bacteria

A Pseudomonas aeruginosa transposon library was subjected to 0 or 1 mM chlorate for 30 minutes. These cultures were sequenced to determine the relative abundance of transposon mutants in under each condition. Using these methods, we identified mutations that conferred a fitness advantage during chlorate exposure, indicating that these mutations increase chlorate resistance in P. aeruginosa.

In particular, a transposon library of P. aeruginosa generated previously (Basta, Bergkessel et al. 2017). was subjected to chlorate exposure.

Three aliquots (three replicates) of the transposon library were thawed on ice, and each diluted into 20 mL LB with 40 mM KNO₃ (final OD₅₀₀=0.05). Cultures were grown anaerobically for 13.5 hours. Under anoxic conditions, each culture was split in half (control sample, chlorate treated sample), pelleted, and washed with LB twice to remove the KNO₃. Cells were pelleted again and resuspended either in LB (control; final OD₅₀₀=2) or LB with 1 mM NaClO₃ (treatment; final OD=2). Cells were incubated anaerobically+/−NaClO₃ for 30 minutes, after which cultures were removed from the anaerobic glove box, pelleted, and wash with LB three times to remove NaClO₃. Resuspension were diluted into 25 mL LB culture at OD₅₀₀=0.02 for control samples and OD₅₀₀=0.04 for chlorate treated samples (assumed ˜50% cell death with chlorate treatment). Cultures were grown aerobically for 4.4±0.3 and 5.4±0.7 doublings for control and chlorate treated samples, respectively (mean±SEM). After aerobic outgrowth, cultures were pelleted and stored at −80° C.

The transposon library was then subjected to sequencing and data analysis. In particular, genomic DNA was extracted from pelleted samples and prepared for Illumina sequencing as described previously (Basta, Bergkessel et al. 2017).). DNA was sequenced using 100 single-end reads on the Illumina HiSeq 2500 platform at the Millard and Muriel Jacobs Genetics and Genomics Laboratory at Caltech. Reads were screened for the last 12 bases of the transposon sequence (TATAAGAGTCAG), and positive reads had the transposon sequence removed and were mapped to the P. aeruginosa UCBPP-PA14 genome using Bowtie 2. featureCounts was used to determine the total number of insertions per gene, and these data were uploaded to Degust (http://degust.erc.monash.edu) for data normalization and statistical analysis using the Voom/Limma Method option. Genes with 3 reads across 5 or more samples (of 6 total samples) were removed from the data.

Mutant strains were then constructed. In particular UCBPP-PA14 markerless gene deletions were constructed by PCR-amplifying 0.5-1 kb fragments immediately upstream and downstream of the target locus (primers used in this study are listed in Table S #). Fragments were Gibson cloned (NEB E2611) ((Gibson 2011) into HindIII- and SacI-digested pMQ30 ((Shanks, Caiazza et al. 2006), and the resulting plasmids were transformed into E. coli DH10B cells using LB supplemented with 20 μg ml⁻¹ gentamicin for selection.

These plasmids were then moved to UCBPP-PA14 via triparental conjugation (with E. coli+pRK2013), and merodiploids were selected by plating on VBMM medium supplemented with 50 μg ml⁻¹ gentamicin. (Choi and Schweizer 2006). Streak-purified merodiploid strains were resuspended in PBS and plated on LB supplemented with 10% sucrose. Plates were incubated at room temperature and sucrose resistant/gentamicin sensitive colonies were PCR-screened to confirm that the genomic region had been deleted.

Chlorate sensitivity assay was then performed. In particular, to test mutants for chlorate sensitivity, overnight cultures of each strain were grown aerobically in LB with 40 mM KNO₃. Cultures were pelleted, washed twice with LB to remove residual KNO₃, and resuspended in LB without (control) or with 1 mM NaClO₃ to final OD₅₀₀=1. Cultures were moved to the anaerobic glove box to adapt to anoxia.

Cultures were incubated at 37° C. for 72 hours, after which they were removed from the anaerobic glove box to determine viable cell counts for calculating percent survival as described previously) (Spero and Newman 2018).). At 72 hours, part of the culture was also pelleted, and supernatant collected for quantifying chlorate concentrations via ion chromatography.

Chlorate quantification was then performed via ion chromatography. In particular, culture samples were pelleted, and the supernatant was diluted 1:10 in dH₂O in a 0.5 mL vial (Thermo Fisher Scientific catalog numbers 038010 and 038011). Chlorate concentrations were determined using a Dionex ICS 2000 ion chromatography as described previously (Spero and Newman 2018).) at the Environmental Analysis Center at Caltech.

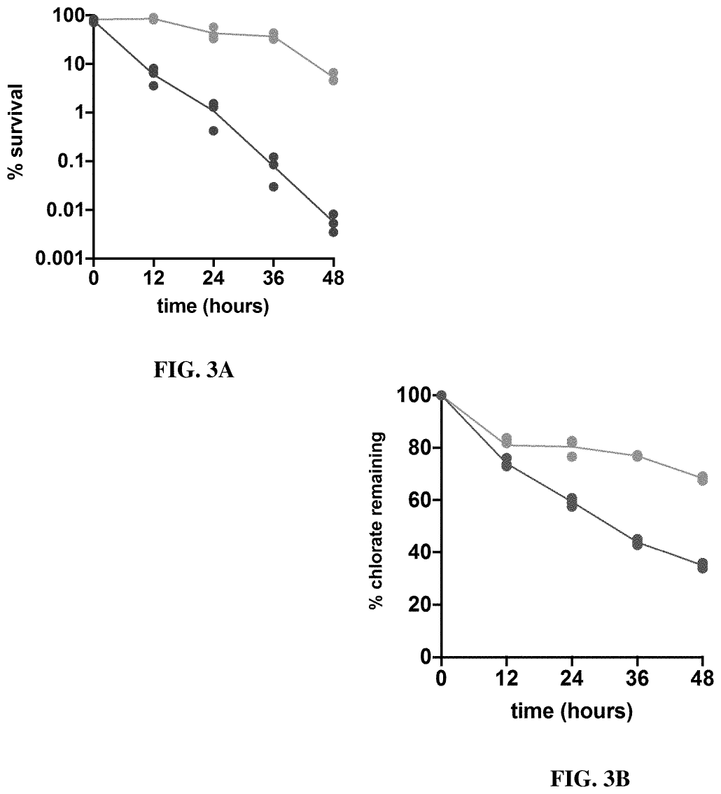

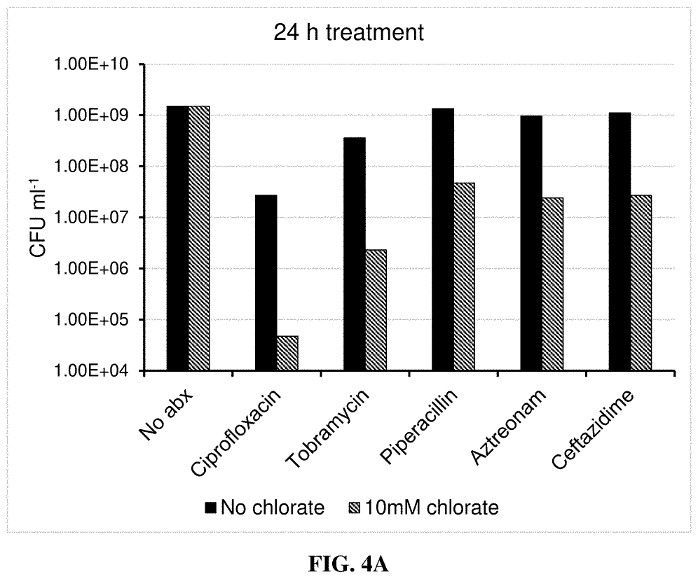

The results shown in FIG. 2A, FIG. 2B, FIG. 3A and FIG. 3B show that mutants such as ΔnarGHJI and B ΔhflC. are more resistant to chlorate than WT, as indicated by higher % survival, and this resistance is correlated with lower rates of Nar activity (lower rates of chlorate reduction indicated as higher % chlorate remaining).

These data support the conclusion that the primary mechanism of chlorate resistance in P. aeruginosa is reduced Nar (nitrate reductase) activity. Reduced Nar activity is likely to be the primary mechanism of chlorate resistance across other Nar-containing, chlorate-sensitive bacteria. Because Nar activity is likely important for pathogen growth and survival within hypoxic/anoxic host environments (e.g. including in chronic wounds), a chlorate resistant mutant with decreased Nar activity will likely have a fitness disadvantage in the host environment. Thus, becoming resistant to chlorate could weaken pathogens such that they cannot survive in those environments, which is expected to be beneficial from a clinical outcome's perspective, as will be understood by a skilled person.

### Example 3: Chlorate Effects on Nar-Containing Bacteria

Plate streaked with P. Aeruginosa WT was grown on LB and after 12 hrs 5 mL culture from a swab of WT was started in LB+40 mM KNO3 and grown 24 hrs at 37 C with shaking at 250 rpm: 6:15 AM.

After 24 hr a humidified chamber was preheated in the 37 C incubator. OD500 of the overnight culture was measured (50 uL O/N in 450 uL LB): 5.360. 1.12 mL of the O/N was added to two Eppendorfs (2.24 mL total) to achieve an GD 2 in 6 mL LB. the eppendorf were spun 5 min at 16000 g.

The supernatant was pipetted off and the pellets resuspended in 1 mL LB (wash). The resuspension was spun 5 min at 16000 g. The supernatant was pipetted off and the pellets resuspended in 1 mL LB. The two resuspensions were transferred to a 50 mL falcon tube with 4 mL LB→6 mL total.

The resuspension was pipetted with P1000 to mix, and 400 uL of the culture were aliquoted into 12 eppendorf tubes.

The following was added to one tube each (antibiotics at 10× MIC):

TABLE 3
Composition

| Base | | |
|---|---|---|
| composition | Tube | Added mixture component |
| Resuspended | LB only | LB only: 20 uL of dH20 |
| P. Aeruginosa | Chlorate only | Chlorate: 20 uL of 200 mM |
| WT Bacterial | | chlorate→10 mM |
| Culture | Cipro only | Ciprofloxacin: 4 uL of 0.5 mg/mL |
| | | cipro (10x dilution of 5 mg/mL |
| | | cipro stock)→5 ug/mL |
| | Piper only | Piperacillin: 25.6 uL of 5 |
| | | mg/mL→320 ug/mL |
| | Ceft only | Ceftazidime: 3.2 uL of 5 |
| | | mg/mL→40 ug/mL |
| | Aztreo only | Aztreonam: 12.8 uL of 5 |
| | | mg/mL→160 ug/mL |
| | Tobra only | Tobramycin: 3.2 uL of 5 |
| | | mg/mL→40 ug/mL |
| | Chlorate + Cipro | 20 uL of 200 mM chlorate, |
| | | 4 uL of 0.5 mg/mL cipro |
| | Chlorate + Piper | 20 uL of 200 mM chlorate, |
| | | 25.6 uL of 5 mg/mL piper |
| | Chlorate + Ceft | 20 uL of 200 mM chlorate, |
| | | 3.2 uL of 5 mg/mL ceft |
| | Chlorate + Aztreo | 20 uL of 200 mM chlorate, |
| | | 12.8 uL of 5 mg/mL aztreo |
| | Chlorate + Tobra | 20 uL of 200 mM chlorate, |
| | | 3.2 uL of 5 mg/mL tobra |

The tubes were vortexed to mix and aliquot 150 uL from each tube were then added into the following wells:

TABLE 4
plate configuration Row B and Row C

| Column | Row B | Row C |
|---|---|---|
| Column 2 | LB only | Tobra only |
| Column 3 | Chlorate only | Chlorate + Cipro |
| Column 4 | Cipro only | Chlorate + Piper |
| Column 5 | Piper only | Chlorate + Ceft |
| Column 6 | Ceft only | Chlorate + Aztreo |
| Column 7 | Aztreo only | Chlorate + Tobra |

The plate (with lid on) were placed in the preheated humidified chamber and incubate 24 hrs at 37 C without shaking: 7 AM. The 12 eppendorf tubes were also incubated 24 hrs at 37 C without shaking.

180 uL of PBS was added to Rows B-C Columns 1-12. After 24 hrs of incubation transfer 100 uL of culture from each well to the following new well (Row A) (pipette to mix before transfer):

TABLE 5
plate configuration INT experiment - Row A

| Column | Row A |
|---|---|
| Column 1 | LB only |
| Column 2 | Chlorate only |
| Column 3 | Cipro only |
| Column 4 | Piper only |
| Column 5 | Ceft only |
| Column 6 | Aztreo only |
| Column 7 | Tobra only |
| Column 8 | Chlorate + Cipro |
| Column 9 | Chlorate + Piper |
| Column 10 | Chlorate + Ceft |
| Column 11 | Chlorate + Aztreo |
| Column 12 | Chlorate + Tobra |

Serial dilutions were made and plate dripped 10 uL of all dilutions on LB at 37 C (10⁰-10⁵). The dilutions and plating were repeated with the Eppendorf cultures!

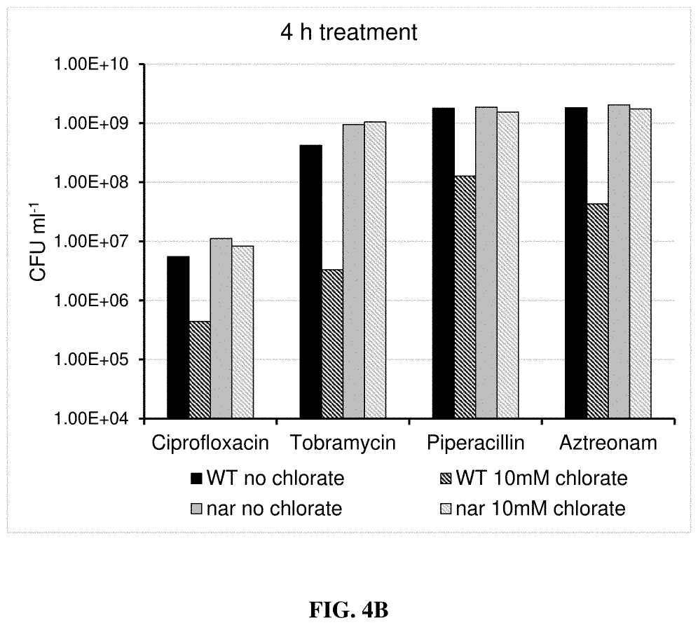

The results shown in FIG. 4A support existence of a synergic effect in the combined use of chlorate and antibiotics in modifying on the viability of the P. aeruginosa WT, and related ability to survive as will be understood by a skilled person.

Experiments were then performed on P. aeruginosa WT to testNnar dependency of the synergic effect. 5 mL culture from a swab of WT and nar in LB+40 mM KNO3 (200 uL of 1M KNO3) were grown 24 hrs at 37 C with shaking at 250 rpm: 7:45 am.

OD500 of the overnight cultures (50 uL O/N in 450 uL LB) was measured and WT: 4.522 Nar: 4.419 were detected.

The following volumes of the O/Ns to two Eppendorfs to achieve an OD 2 in 6 mL LB were added WT: 1.325 mL (2.65 mL total) and Nar: 1.358 mL (2.7155 mL total)

The volume was Spunn 5 min at 16000 g, the supernatant was pipetted off and the pellets were resuspended in 1 mL LB (wash). The resuspension was spun 5 min at 16000 g.

The supernatant was pipetted off and the pellets resuspend in 1 mL LB, the resuspensions were transferred to 50 mL falcon tubes with 4 mL LB→6 mL total and vortexed to mix.

400 uL of each culture was aliquoted into 6 eppendorf tubes (12 tubes total) and the following was added to one tube of each strain (antibiotics at 10× MIC):

TABLE 6
Composition

| Base | | |
|---|---|---|
| composition | Tube | Added mixture component |
| Resuspended | LB only | LB only: 20 uL of dH20 |
| P. Aeruginosa | Chlorate only | Chlorate: 20 uL of 200 mM |
| WT Bacterial | | chlorate→10 mM |
| Culture | Cipro only | Ciprofloxacin: 4 uL of 0.5 mg/mL |
| | | cipro (10x dilution of 5 mg/mL |
| | | cipro stock)→5 ug/mL |
| | Tobra only | Tobramycin: 3.2 uL of 5 |
| | | mg/mL→40 ug/mL |
| | Chlorate + Cipro | 20 uL of 200 mM chlorate, |
| | | 4 uL of 0.5 mg/mL cipro |
| | Chlorate + Tobra | 20 uL of 200 mM chlorate, |
| | | 3.2 uL of 5 mg/mL tobra |

The tubes were vortexed to mix and incubated the tubes 4 hrs at 37 C without shaking (8:30 am-12:30 pm) 180 uL of PBS were added to Rows B-F Columns 1-12 in a 96-well plate.

After 4 hrs of incubation each tube was vortexed. 200 uL of each strain culture was transferred from each tube to the following well in Row A according to the following configuration.

TABLE 7
plate configuration WT/Nar experiments - Row A

| Column | Strain | Row A |
|---|---|---|
| Column 1 | WT | LB only |
| Column 2 | WT | Chlorate only |
| Column 3 | WT | Cipro only |
| Column 4 | WT | Tobra only |
| Column 5 | WT | Chlorate + Cipro |
| Column 6 | WT | Chlorate + Tobra |
| Column 7 | Nar | LB only |
| Column 8 | Nar | Chlorate only |
| Column 9 | Nar | Cipro only |
| Column 10 | Nar | Tobra only |
| Column 11 | Nar | Chlorate + Cipro |
| Column 12 | Nar | Chlorate + Tobra |

Serial dilutions and drip plate were made 10 uL of all dilutions on LB agar at 37 C (10⁰-10⁵).

The results shown in FIG. 4B confirmed the synergic effect shown for all combinations of chlorate and antibiotic detected in P. aeruginosa WT and but not for the treatment of nar mutated bacteria in all the experiments performed.

Since Nar containing bacteria are part of chronic wound bacterial communities, these results further support the conclusion that combined chlorate and antibiotic treatment at antibiotic therapeutic concentrations is expected to be synergistically effective in reducing viability up to killing the bacteria and dismantling the biofilm thus supporting resolution of the chronicity of a wound.

In particular, the above data support the conclusions that chlorate and antibiotics can act synergistically by targeting distinct bacteria populations, and in particular cells with or without access to oxygen, to substantially prevent and disrupt biofilm aggregate as also exemplified in Examples 5-6 of U.S. Ser. No. 16/157,885 filed on Oct. 11, 2018 published as US 2019/0142864.

The synergic ability shown by the above results further supports a conclusion of effectiveness of the combined administration of antibiotic administered at a therapeutic concentrations in treating any further additional infection of Nar-containing bacteria, inside or outside wounds of a tissue or an organ such as treatment of infection by Nar-containing bacteria in lungs, ear and eye as well as in the gastrointestinal tract of an individual, as will be understood by a skilled person.

In summary, described herein are wound healing matrices, compounds, compositions, methods and systems comprising one or more chlorates, one or more chlorites, one or more antibiotics, one or more other antimicrobials, and/or one or more wound healing agents.

The examples set forth above are provided to give those of ordinary skill in the art a complete disclosure and description of how to make and use the embodiments of the materials, compounds, compositions, systems and methods of the disclosure, and are not intended to limit the scope of what the inventors regard as their disclosure. All patents and publications mentioned in the specification are indicative of the levels of skill of those skilled in the art to which the disclosure pertains.

The entire disclosure of each document cited (including patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence. Further, the computer readable form of the sequence listing of the ASCII text file P2493-US-Seq-List_ST25 is incorporated herein by reference in its entirety.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by preferred embodiments, exemplary embodiments and optional features, modification and variation of the concepts herein disclosed can be resorted to by those skilled in the art upon the reading of the present disclosure, and that such modifications and variations are considered to be within the scope of this disclosure as defined by the appended claims.

It is also to be understood that the terminology used herein is for the purpose of describing particular embodiments only and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified can be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all sub-ranges, as well as, all individual values included in the ranges given are intended to be included in the disclosure. Any one or more individual members of a range or group disclosed herein can be excluded from a claim of this disclosure. The disclosure illustratively described herein suitably can be practiced in the absence of any element or elements, limitation or limitations which are not specifically disclosed herein.

A number of embodiments of the disclosure have been described. The specific embodiments provided herein are examples of useful embodiments of the disclosure and it will be apparent to one skilled in the art that the disclosure can be carried out using a large number of variations of the devices, device components, methods steps set forth in the present description. As will be obvious to one of skill in the art, methods and devices useful for the present methods can include a large number of optional composition and processing elements and steps.

In particular, it will be understood that various modifications may be made without departing from the spirit and scope of the present disclosure. Accordingly, other embodiments are within the scope of the following claims.

### REFERENCES

(1988 (November), 1998 (May), 2007 (February)). The Annual UK Drug Tariff. The Stationary Office. London, UK, The UK Drug Tariff.
(2011). “Growth factor” at Dorland's Medical Dictionary.” from https:/web.archive.org/web/20110607011425/http://www.mercksource.com/pp/us/cns/cns_hl_dorlands_split.jsp?pg=/ppdocs/us/common/dorlands/dorland/three/000038554.htm
Adamson, B., et al. (1996). “Delayed repair: the role of glutathione in a rat incisional wound model.” Journal of Surgical Research 62(2): 159-164.
Ågren, M. S. (1990). “Studies on zinc in wound healing.” Acta Derm Venereol Suppl 154: 1-36.
Ågren, M. S., et al. (2006). “A randomized, double-blind, placebo-controlled multicenter trial evaluating topical zinc oxide for acute open wounds following pilonidal disease excision.” Wound repair and regeneration 14(5): 526-535.
Aihara, M., et al. (2000). “Effects of N-acetylcysteine and ambroxol on the production of IL-12 and IL-10 in human alveolar macrophages.” Respiration 67(6): 662-671.
Altschul S F, M. T., Schaffer A A, Zhang J, Zhang Z, Miller W, Lipman D J. (1997). “Gapped BLAST and PSI-BLAST: a new generation of protein database search programs.” Nucleic Acids Res. 25(17): 14.
Alvarez-Suarez, J. M., et al. (2014). “The composition and biological activity of honey: a focus on Manuka honey.” Foods 3(3): 420-432.
Armstrong, S. and C. Ruckley (1997). “Use of a fibrous dressing in exuding leg ulcers.” Journal of Wound Care 6(7): 322-324.
BA, M. (1999). The Skin Philadelphia, Pa., Saunders.
Bao, P., et al. (2009). “The role of vascular endothelial growth factor in wound healing.” Journal of Surgical Research 153(2): 347-358.
Barañano, D. E., et al. (2002). “Biliverdin reductase: a major physiologic cytoprotectant.” Proceedings of the National Academy of Sciences 99(25): 16093-16098.
Basta, D. W., et al. (2017). “Identification of fitness determinants during energy-limited growth arrest in Pseudomonas aeruginosa.” MBio 8(6).
Baumann, L. S. and J. S. Md (1999). “The effects of topical vitamin E on the cosmetic appearance of scars.” Dermatologic Surgery 25(4): 311-315.
Beck, L. S., et al. (1993). “One systemic administration of transforming growth factor-beta 1 reverses age- or glucocorticoid-impaired wound healing.” The Journal of clinical investigation 92(6): 2841-2849.
Bernard, G., et al. (1984). “Effect of N-acetylcysteine on the pulmonary response to endotoxin in the awake sheep and upon in vitro granulocyte function.” The Journal of clinical investigation 73(6): 1772-1784.
Boateng, J. S., et al. (2008). “Wound healing dressings and drug delivery systems: a review.” Journal of pharmaceutical sciences 97(8): 2892-2923.
Bolton, L. and L. Van Rijswijk (1991). “Wound dressings: meeting clinical and biological needs.” Dermatology nursing 3(3): 146-161.
Calabrese, V., et al. (2008). “Curcumin and the cellular stress response in free radical-related diseases.” Molecular nutrition & food research 52(9): 1062-1073.
Cano Sanchez, M., et al. (2018). “Targeting oxidative stress and mitochondrial dysfunction in the treatment of impaired wound healing: a systematic review.” Antioxidants 7(8): 98.
Chedid, M., et al. (1996). “Glucocorticoids inhibit keratinocyte growth factor production in primary dermal fibroblasts.” Endocrinology 137(6): 2232-2237.
Chen, W. J., et al. (1992). “Characterization of biologic properties of wound fluid collected during early stages of wound healing.” Journal of Investigative Dermatology 99(5): 559-564.
Chigurupati, S., et al. (2010). “A synthetic uric acid analog accelerates cutaneous wound healing in mice.” PloS one 5(4): e10044.
Choi, K.-H. and H. P. Schweizer (2006). “mini-Tn 7 insertion in bacteria with single att Tn 7 sites: example Pseudomonas aeruginosa.” Nature protocols 1(1): 153.
Choi, S. M., et al. (2018). “Effects of structurally stabilized EGF and bFGF on wound healing in type I and type II diabetic mice.” Acta biomaterialia 66: 325-334.
Choi, S. M., et al. (2016). “Development of stabilized growth factor-loaded hyaluronate-collagen dressing (HCD) matrix for impaired wound healing.” Biomaterials research 20(9): 1-7.
Chu, H.-q., et al. (2006). “Aminoglycoside ototoxicity in three murine strains and effects on NKCC1 of stria vascularis.” Chinese medical journal 119(12): 980-985.
Corkery, K. (2000). “Inhalable drugs for systemic therapy.” Respiratory care 45(7): 831-835.
Cotgreave, I., et al. (1991). “The metabolism of N-acetylcysteine by human endothelial cells.” Biochemical pharmacology 42(1): 13-16.
D'Argenio, D. A., et al. (2007). “Growth phenotypes of Pseudomonas aeruginosa lasR mutants adapted to the airways of cystic fibrosis patients.” Mol Microbiol 64(2): 512-533.
da Rocha, R. P., et al. (2002). “Effects of a vitamin pool (vitamins A, E, and C) on the tissue necrosis process: experimental study on rats.” Aesthetic plastic surgery 26(3): 197-202.
Dealey, C. (1993). “Role of hydrocolloids in wound management.” British journal of nursing 2(7): 358-365.
Dhall, S., et al. (2014). “Generating and reversing chronic wounds in diabetic mice by manipulating wound redox parameters.” Journal of diabetes research 2014: 18.
D M, C. (1999). “Wound healing: New understandings.” Nurs Pract Forum 10: 74-86.
Eaglstein, W. H., et al. (1988). “Optimal use of an occlusive dressing to enhance healing: Effect of delayed application and early removal on wound healing.” Archives of dermatology 124(3): 392-395.
Ehrlich, H. P., et al. (1973). “Effects of vitamin A and glucocorticoids upon inflammation and collagen synthesis.” Annals of surgery 177(2): 222-227.
Elçin, Y. M., et al. (2001). “Extensive in vivo angiogenesis following controlled release of human vascular endothelial cell growth factor: implications for tissue engineering and wound healing.” Artificial organs 25(7): 558-565.
Falabella, A. F. (2006). “Debridement and wound bed preparation.” Dermatologic therapy 19(6): 317-325.
Fang, R. C. and R. D. Galiano (2008). “A review of becaplermin gel in the treatment of diabetic neuropathic foot ulcers.” Biologics: targets & therapy 2(1): 1-12.
Ferreira, M. C., et al. (2006). “Complex wounds.” Clinics 61(6): 571-578.
Fitzmaurice, S., et al. (2011). “Antioxidant therapies for wound healing: a clinical guide to currently commercially available products.” Skin pharmacology and physiology 24(3): 113-126.
Flanigan, K. H. (1997). “Nutritional aspects of wound healing.” Advances in wound care: the journal for prevention and healing 10(3): 48-52.
Fukunaga, A., et al. (2006). “Our method of povidone-iodine ointment and gauze dressings reduced catheter-related infection in serious cases.” Dermatology 212(Suppl. 1): 47-52.
Garcia-Orue, I., et al. (2017). “Novel nanofibrous dressings containing rhEGF and Aloe vera for wound healing applications.” International journal of pharmaceutics 523(2): 556-566.
Gibson, D. G. (2011). “Enzymatic assembly of overlapping DNA fragments.” Methods in enzymology 498: 349-361.
Gilliland, E. L., et al. (1988). “Bacterial colonisation of leg ulcers and its effect on the success rate of skin grafting.” Annals of the Royal College of Surgeons of England 70(2): 105-108.
GM, E. (2007). Wound dressings. UK, Churchill Livingstone.
Gökşen, S., et al. (2017). “Application of platelet derived growth factor-BB and diabetic wound healing: the relationship with oxidative events.” Free radical research 51(5): 498-505.
Gomes, F., et al. (2012). “Farnesol in combination with N-acetylcysteine against Staphylococcus epidermidis planktonic and biofilm cells.” Brazilian Journal of Microbiology 43(1): 235-242.
Grandjean, E. M., et al. (2000). “Efficacy of oral long-term N-acetylcysteine in chronic bronchopulmonary disease: a meta-analysis of published double-blind, placebo-controlled clinical trials.” Clinical therapeutics 22(2): 209-221.
Greenhalgh, D. G. (1996). “The role of growth factors in wound healing.” Journal of Trauma and Acute Care Surgery 41(1): 159-167.
Greenhalgh, D. G., et al. (1990). “PDGF and FGF stimulate wound healing in the genetically diabetic mouse.” The American journal of pathology 136(6): 1235-1246.
G S, S. (1999). Molecular regulation of wound healing. St. Louis, Mo., Mosby.
Gu, D.-l., et al. (2004). “Adenovirus encoding human platelet-derived growth factor-B delivered in collagen exhibits safety, biodistribution, and immunogenicity profiles favorable for clinical use.” Molecular therapy 9(5): 699-711.
H, O. (1995). Nutrients in profile. 2nd edition. Queensland, Kelvin Grove. Hanschmann, E.-M., et al. (2013). “Thioredoxins, glutaredoxins, and peroxiredoxins—molecular mechanisms and health significance: from cofactors to antioxidants to redox signaling.” Antioxidants & redox signaling 19(13): 1539-1605.
Harding, K., et al. (2002). “Clinical review Healing chronic wounds.” Br Med J 324: 160-163.
Hareendran, A., et al. (2005). “Measuring the impact of venous leg ulcers on quality of life.” Journal of Wound Care 14(2): 53-57.
Hart, A., et al. (2004). “Sensory neuroprotection, mitochondrial preservation, and therapeutic potential of N-acetyl-cysteine after nerve injury.” Neuroscience 125(1): 91-101.
Hemilä, H. and R. M. Douglas (1999). “Vitamin C and acute respiratory infections.” The international journal of tuberculosis and lung disease 3(9): 756-761.
Heydorn, A., et al. (2000). “Quantification of biofilm structures by the novel computer program COMSTAT.” Microbiology 146(10): 2395-2407.
Hoffman, L. R., et al. (2009). “Pseudomonas aeruginosa lasR mutants are associated with cystic fibrosis lung disease progression.” J Cyst Fibros 8(1): 66-70.
Hoffman, L. R., et al. (2010). “Nutrient availability as a mechanism for selection of antibiotic tolerant Pseudomonas aeruginosa within the CF airway.” PLoS Pathog 6(1): e1000712.
Hofmann, B., et al. (2002). “Peroxiredoxins.” Biological chemistry 383(3-4): 347-364.
Hudspith, J. and S. Rayatt (2004). “First aid and treatment of minor burns.” Bmj 328(7454): 1487-1489.
Hussain, Z., et al. (2017). “Exploring recent developments to improve antioxidant, anti-inflammatory and antimicrobial efficacy of curcumin: A review of new trends and future perspectives.” Materials science and engineering: C 77: 1316-1326.
Jagetia, G. C. and G. Rajanikant (2004). “Role of curcumin, a naturally occurring phenolic compound of turmeric in accelerating the repair of excision wound, in mice whole-body exposed to various doses of 7-radiation.” Journal of Surgical Research 120(1): 127-138.
Jiang, T., et al. (2014). “Nrf2 suppresses lupus nephritis through inhibition of oxidative injury and the NF-κB-mediated inflammatory response.” Kidney international 85(2): 333-343.
Johnson L S, E. S., Portugaly E (2010). “Hidden Markov model speed heuristic and iterative HMM search procedure.” BMC Bioinformatics 11(431): 8.
Judith, R., et al. (2010). “Application of a PDGF-containing novel gel for cutaneous wound healing.” Life sciences 87(1-2): 1-8.
Jull, A. B., et al. (2015). “Honey as a topical treatment for wounds.” Cochrane Database of Systematic Reviews(3): Art. No.: CD005083.
Karlin, S. and S. F. Altschul (1990). “Methods for assessing the statistical significance of molecular sequence features by using general scoring schemes.” Proceedings of the National Academy of Sciences 87(6): 2264-2268.
Karlin, S. and S. F. Altschul (1993). “Applications and statistics for multiple high-scoring segments in molecular sequences.” Proceedings of the National Academy of Sciences 90(12): 5873-5877.
Khalaf, A., et al. (2015). “Antibacterial, anti-biofilm activity of some non-steroidal anti-inflammatory drugs and N-acetyl cysteine against some biofilm producing uropathogens.” American Journal of Epidemiology 3(1): 1-9.
Khan, R. U., et al. (2017). “Towards a better understanding of the therapeutic applications and corresponding mechanisms of action of honey.” Environmental Science and Pollution Research 24(36): 27755-27766.
Kietzmann, M. and M. Braun (2006). “Effects of the zinc oxide and cod liver oil containing ointment Zincojecol in an animal model of wound healing.” DTW. Deutsche tierarztliche Wochenschrift 113(9): 331-334.
Kirschmann G, K. J. (1996). Nutrition almanac, 4th edition. New York, N.Y., McGraw-Hill.
Komarcević, A. (2000). “The modern approach to wound treatment.” Medicinski pregled 53(7-8): 363-368.
Krasner, D., et al. (1993). “The ABCs of wound care dressings.” wound management 66: 68-69.
Kümin, A., et al. (2006). “Peroxiredoxin 6 is a potent cytoprotective enzyme in the epidermis.” The American journal of pathology 169(4): 1194-1205.
Kundukad, B., et al. (2017). “Mechanistic action of weak acid drugs on biofilms.” Scientific reports 7(1): 1-12.
Lansdown, A. B. (2002). “Metallothioneins: potential therapeutic aids for wound healing in the skin.” Wound repair and regeneration 10(3): 130-132.
Lazarus, G. S., et al. (1994). “Definitions and guidelines for assessment of wounds and evaluation of healing.” Arch Dermatol 130(4): 489-493.
Lee, A.-R. C., et al. (2005). “Reversal of silver sulfadiazine-impaired wound healing by epidermal growth factor.” Biomaterials 26(22): 4670-4676.
Liao, Z., et al. (2006). “Multi-center clinical study of the effect of silver nitrate ointment on the partial-thickness burn wounds.” Zhonghua shao shang za zhi=Zhonghua shaoshang zazhi=Chinese journal of burns 22(5): 359-361.
Lin, Y.-J., et al. (2015). “Stimulation of wound healing by PU/hydrogel composites containing fibroblast growth factor-2.” Journal of Materials Chemistry B 3(9): 1931-1941.
Lindberg, T., et al. (2015). “A systematic review and meta-analysis of dressings used for wound healing: the efficiency of honey compared to silver on burns.” Contemporary nurse 51(2-3): 121-134.
Linder, M. C. (1991). Nutritional biochemistry and metabolism: with clinical applications, 2nd edition. East Norwalk, Conn., Appleton & Lange
Long, D. W., et al. (2017). “Controlled delivery of platelet-derived proteins enhances porcine wound healing.” Journal of Controlled Release 253: 73-81.
Long, M., et al. (2016). “An essential role of NRF2 in diabetic wound healing.” Diabetes 65(3): 780-793.
Loo, A. E. K., et al. (2012). “Effects of hydrogen peroxide on wound healing in mice in relation to oxidative damage.” PloS one 7(11): e49215.
Lord, M. S., et al. (2017). “Perlecan and vascular endothelial growth factor-encoding DNA-loaded chitosan scaffolds promote angiogenesis and wound healing.” Journal of Controlled Release 250: 48-61.
Lu, J. and A. Holmgren (2014). “The thioredoxin antioxidant system.” Free Radical Biology and Medicine 66: 75-87.
Lubbers, J. and J. Bianchine (1984). “Effects of the acute rising dose administration of chlorine dioxide, chlorate and chlorite to normal healthy adult male volunteers.” Journal of environmental pathology, toxicology and oncology: official organ of the International Society for Environmental Toxicology and Cancer 5(4-5): 215-228.
Lubbers, J., et al. (1984). “The effects of chronic administration of chlorine dioxide, chlorite and chlorate to normal healthy adult male volunteers.” Journal of environmental pathology, toxicology and oncology: official organ of the International Society for Environmental Toxicology and Cancer 5(4-5): 229-238.
Lubbers, J., et al. (1984). “The effects of chronic administration of chlorite to glucose-6-phosphate dehydrogenase deficient healthy adult male volunteers.” Journal of environmental pathology, toxicology and oncology: official organ of the International Society for Environmental Toxicology and Cancer 5(4-5): 239-242.
Lubbers, J. R., et al. (1982). “Controlled clinical evaluations of chlorine dioxide, chlorite and chlorate in man.” Environmental health perspectives 46: 57-62.
Lubbers, J. R., et al. (1981). “Controlled clinical evaluations of chlorine dioxide, chlorite and chlorate in man.” Fundamental and applied Toxicology 1(4): 334-338.
Mandla, S., et al. (2018). “Multimodal bioactive material approaches for wound healing.” APL bioengineering 2(2): 021503.
Mann, A., et al. (2006). Granulocyte-macrophage colony-stimulating factor is essential for normal wound healing. Journal of Investigative Dermatology Symposium Proceedings, Elsevier.
Marchese, A., et al. (2003). “Effect of fosfomycin alone and in combination with N-acetylcysteine on E. coli biofilms.” International journal of antimicrobial agents 22: S95-S100.
Marczin, N., et al. (2003). “Antioxidants in myocardial ischemia-reperfusion injury: therapeutic potential and basic mechanisms.” Archives of Biochemistry and Biophysics 420(2): 222-236.
Martin, P. (1997). “Wound healing—aiming for perfect skin regeneration.” Science 276(5309): 75-81.
Martins-Green (2020). Wound Healing, Tissue Repair, and Regeneration in Diabetes 9780128164136.
Mayet, N., et al. (2014). “A comprehensive review of advanced biopolymeric wound healing systems.” Journal of pharmaceutical sciences 103(8): 2211-2230.
Miller, M. S. (1999). “Use of topical recombinant human platelet-derived growth factor-BB (becaplermin) in healing of chronic mixed arteriovenous lower extremity diabetic ulcers.” The Journal of foot and ankle surgery 38(3): 227-231.
Misra, A. and J. Nanchahal (2003). “Use of gauze soaked in povidone iodine for dressing acute open wounds.” Plastic and Reconstructive Surgery 111(6): 2105-2106.
Mohanty, C. and S. K. Sahoo (2017). “Curcumin and its topical formulations for wound healing applications.” Drug Discovery Today 22(10): 1582-1592.
Moore, K., et al. (2006). “Prediction and monitoring the therapeutic response of chronic dermal wounds.” International wound journal 3(2): 89-98.
Moreno-Vivian, C., et al. (1999). “Prokaryotic nitrate reduction: molecular properties and functional distinction among bacterial nitrate reductases.” J Bacteriol 181(21): 6573-6584.
Motta, G. J., et al. (2004). “Impact of antimicrobial gauze on bacterial colonies in wounds that require packing.” Ostomy/wound management 50(8): 48-62.
Mudge, B. P., et al. (2002). “Role of glutathione redox dysfunction in diabetic wounds.” Wound repair and regeneration 10(1): 52-58.
Mukai, K., et al. (2016). “Effectiveness of Changing the Application of Japanese Honey to a Hydrocolloid Dressing in Between the Inflammatory and Proliferative Phases on Cutaneous Wound Healing in Male Mice.” Wounds: a compendium of clinical research and practice 29(1): 1-9.
Murray, R. Z., et al. (2019). “Development and use of biomaterials as wound healing therapies.” Burns & trauma 7(7:2).
Musalmah, M., et al. (2005). “Comparative effects of palm vitamin E and α-tocopherol on healing and wound tissue antioxidant enzyme levels in diabetic rats.” Lipids 40(6): 575-580.
Myers, E. W. and W. Miller (1988). “Optimal alignments in linear space.” Computer applications in the biosciences: CABIOS 4(1): 11-17.
Naradzay, F. and R. Alson (2005). “Burns, thermal.” Web MD.
Needleman, S. B. and C. D. Wunsch (1970). “A general method applicable to the search for similarities in the amino acid sequence of two proteins.” Journal of molecular biology 48(3): 443-453.
Nery, R. A., et al. (2015). “Uric acid and tissue repair.” ABCD. Arquivos Brasileiros de Cirurgia Digestiva (São Paulo) 28(4): 290-292.
Niaz, K., et al. (2017). “Health benefits of manuka honey as an essential constituent for tissue regeneration.” Current drug metabolism 18(10): 881-892.
Panchatcharam, M., et al. (2006). “Curcumin improves wound healing by modulating collagen and decreasing reactive oxygen species.” Molecular and cellular biochemistry 290(1): 87-96.
Patel, G. K. (2005). “The role of nutrition in the management of lower extremity wounds.” The international journal of lower extremity wounds 4(1): 12-22.
Paterson, R. L., et al. (2003). “The effect of N-acetylcysteine on nuclear factor-KB activation, interleukin-6, interleukin-8, and intercellular adhesion molecule-1 expression in patients with sepsis.” Critical care medicine 31(11): 2574-2578.
Patrick, B. N., et al. (2006). “Acute renal failure associated with vancomycin- and tobramycin-laden cement in total hip arthroplasty.” Annals of Pharmacotherapy 40(11): 2037-2042.
Pearson W R, L. D. (1988). “Improved tools for biological sequence comparison.” Proc Natl Acad Sci USA 85(8): 5.
Pearson, W. R. and D. J. Lipman (1988). “Improved tools for biological sequence comparison.” Proceedings of the National Academy of Sciences 85(8): 2444-2448.
Percival, N. J. (2002). “Classification of wounds and their management.” Surgery (Oxford) 20(5): 114-117.
Peshenko, I. V. and H. Shichi (2001). “Oxidation of active center cysteine of bovine 1-Cys peroxiredoxin to the cysteine sulfenic acid form by peroxide and peroxynitrite.” Free Radical Biology and Medicine 31(3): 292-303.
Petersen Shay, K., et al. (2008). “Is α-lipoic acid a scavenger of reactive oxygen species in vivo?Evidence for its initiation of stress signaling pathways that promote endogenous antioxidant capacity.” IUBMB life 60(6): 362-367.
Pierce, G., et al. (1991). “Platelet-derived growth factor-BB and transforming growth factor beta 1 selectively modulate glycosaminoglycans, collagen, and myofibroblasts in excisional wounds.” The American journal of pathology 138(3): 629.
Pierce, G. F., et al. (1989). “Transforming growth factor beta reverses the glucocorticoid-induced wound-healing deficit in rats: possible regulation in macrophages by platelet-derived growth factor.” Proceedings of the National Academy of Sciences 86(7): 2229-2233.
Purner S K, B. M. (2000). “Collagen based dressings—a review.” Burns 26(1): 54-62.
Queen, D., et al. (2004). “A dressing history.” International wound journal 1(1): 59-77.
Raftos, J. E., et al. (2007). “Kinetics of uptake and deacetylation of N-acetylcysteine by human erythrocytes.” The international journal of biochemistry & cell biology 39(9): 1698-1706.
Ram, M., et al. (2014). “Antioxidant potential of bilirubin-accelerated wound healing in streptozotocin-induced diabetic rats.” Naunyn-Schmiedeberg's archives of pharmacology 387(10): 955-961.
Ram, M., et al. (2016). “Bilirubin modulated cytokines, growth factors and angiogenesis to improve cutaneous wound healing process in diabetic rats.” International immunopharmacology 30: 137-149.
Rasik, A. M. and A. Shukla (2000). “Antioxidant status in delayed healing type of wounds.” International journal of experimental pathology 81(4): 257-263.
Rojas, A. I. and T. J. Phillips (1999). “Patients with chronic leg ulcers show diminished levels of vitamins A and E, carotenes, and zinc.” Dermatologic Surgery 25(8): 601-604.
Rothe, M. and V. Falanga (1989). “Growth factors: their biology and promise in dermatologic diseases and tissue repair.” Archives of dermatology 125(10): 1390-1398.
Russell, L. (2000). “Understanding physiology of wound healing and how dressings help.” British journal of nursing 9(1): 10-21.
Saghazadeh, S., et al. (2018). “Drug delivery systems and materials for wound healing applications.” Advanced drug delivery reviews 127: 138-166.
Schäfer, M. and S. Werner (2008). “Oxidative stress in normal and impaired wound repair.” Pharmacological research 58(2): 165-171.
Schreml, S., et al. (2014). “Luminescent dual sensors reveal extracellular pH-gradients and hypoxia on chronic wounds that disrupt epidermal repair.” Theranostics 4(7): 721.
Sen, C. K. and S. Roy (2008). “Redox signals in wound healing.” Biochimica et Biophysica Acta (BBA)-General Subjects 1780(11): 1348-1361.
Shakespeare, P. (2001). “Burn wound healing and skin substitutes.” Burns 27(5): 517-522.
Shanks, R. M., et al. (2006). “Saccharomyces cerevisiae-based molecular tool kit for manipulation of genes from gram-negative bacteria.” Applied and environmental microbiology 72(7): 5027-5036.
Shukla, A., et al. (1997). “Depletion of reduced glutathione, ascorbic acid, vitamin E and antioxidant defence enzymes in a healing cutaneous wound.” Free radical research 26(2): 93-101.
Smiell, J. M., et al. (1999). “Efficacy and safety of becaplermin (recombinant human platelet-derived growth factor-BB) in patients with nonhealing, lower extremity diabetic ulcers: a combined analysis of four randomized studies.” Wound repair and regeneration 7(5): 335-346.
Smith, E. E., et al. (2006). “Genetic adaptation by Pseudomonas aeruginosa to the airways of cystic fibrosis patients.” Proc Natl Acad Sci USA 103(22): 8487-8492.
Smith, T. F. and M. S. Waterman (1981). “Comparison of biosequences.” Advances in applied mathematics 2(4): 482-489.
Smith T F, W. M. (1981). “Identification of common molecular subsequences.” J Mol Biol 147(1): 3.
Spero, M. A. and D. K. Newman (2018). “Chlorate specifically targets oxidant-starved, antibiotic-tolerant populations of Pseudomonas aeruginosa biofilms.” MBio 9(5): 1-16
Steenfos, H. H. (1994). “Growth factors and wound healing.” Scandinavian journal of plastic and reconstructive surgery and hand surgery 28(2): 95-105.
Strodtbeck, F. (2001). “Physiology of wound healing.” Newborn Infants Nurs Rev 1: 43-52.
Suzuki, T. and M. Yamamoto (2017). “Stress-sensing mechanisms and the physiological roles of the Keap1-Nrf2 system during cellular stress.” Journal of Biological Chemistry 292(41): 16817-16824.
Taylor, R. and T. James (2005). The role of oxidative stress in the development and persistence of pressure ulcers. Pressure ulcer research, Springer: 205-232.
Taylor, T., et al. (1974). “Ascorbic acid supplementation in the treatment of pressure-sores.” The Lancet 304(7880): 544-546.
Ten Dijke, P. and K. K. Iwata (1989). “Growth factors for wound healing.” Bio/Technology 7(8): 793-798.
Tenhunen, R., et al. (1968). “The enzymatic conversion of heme to bilirubin by microsomal heme oxygenase.” Proceedings of the National Academy of Sciences of the United States of America 61(2): 748-755.
ter Riet, G., et al. (1995). “Randomized clinical trial of ascorbic acid in the treatment of pressure ulcers.” Journal of clinical epidemiology 48(12): 1453-1460.
Tsai, M.-L., et al. (2014). “Topical N-acetylcysteine accelerates wound healing in vitro and in vivo via the PKC/Stat3 pathway.” International journal of molecular sciences 15(5): 7563-7578.
Uruno, A., et al. (2015). “The Keap1-Nrf2 system and diabetes mellitus.” Archives of Biochemistry and Biophysics 566: 76-84.
Van Rijswijk, L. (2006). “Ingredient-based wound dressing classification: a paradigm that is passe and in need of replacement.” Journal of Wound Care 15(1): 11-14.
Vervaart, P. and K. Knight (1996). “Oxidative stress and the cell.” Clinical Biochemist Reviews 17: 3-16.
Wallace, E. (1994). “Feeding the wound: nutrition and wound care.” British journal of nursing 3(13): 662-667.
Wawrzyńska, E. and D. Kubies (2018). “Alginate matrices for protein delivery—a short review.” Physiological research 67 (Suppl. 2): S319-S334.
Wiseman, H. (1993). “Vitamin D is a membrane antioxidant Ability to inhibit iron-dependent lipid peroxidation in liposomes compared to cholesterol, ergosterol and tamoxifen and relevance to anticancer action.” FEBS letters 326(1-3): 285-288.
Wlaschek, M. and K. Scharffetter-Kochanek (2005). “Oxidative stress in chronic venous leg ulcers.” Wound repair and regeneration 13(5): 452-461.
Wood, Z. A., et al. (2003). “Structure, mechanism and regulation of peroxiredoxins.” Trends in biochemical sciences 28(1): 32-40.
W R, P. (1991). “Searching protein sequence libraries: comparison of the sensitivity and selectivity of the Smith-Waterman and FASTA algorithms.” Genomics 11(3): 16.
Yang, C., et al. (2013). “Rapamycin and mTOR inhibitors probably have therapeutic effects for post-operative cognitive dysfunction.” Medical hypotheses 81(3): 487-488.
Yang, H., et al. (2001). “Effect of improved topical agents on healing time of deep second-degree burn wound.” Zhongguo xiu fu chong jian wai ke za zhi=Zhongguo xiufu chongjian waike zazhi=Chinese journal of reparative and reconstructive surgery 15(3): 162-164.
Yoshida, T., et al. (2005). “The involvement of thioredoxin and thioredoxin binding protein-2 on cellular proliferation and aging process.” ANNALS-NEW YORK ACADEMY OF SCIENCES 1055: 1-12.
Zheng, H., et al. (2011). “Therapeutic potential of Nrf2 activators in streptozotocin-induced diabetic nephropathy.” Diabetes 60(11): 3055-3066.

1. A wound healing composition for treating and/or preventing chronic wound comprises one or more chlorates alone or in combination with one or more antibiotics, one or more other antimicrobials, and/or one or more wound healing agents in an amount suitable to reduce antibiotic resistance, viability and/or survivability of bacteria in the wound.

2. The wound healing composition of claim 1, wherein the chlorate is in an amount between 1 mM and 200 mM.

3. The wound healing composition of claim 1, wherein the antibiotics comprises amoxicillin, clavulanic acid, clindamycin, aminoglycosides, ciprofloxacin, cefalosporines, and/or metronidazole.

4. The wound healing composition of claim 1, wherein the antibiotic comprises one or more of Ciprofloxacin, Piperacillin, Ceftazidime, Aztreonam, and/or Tobramycin.

5. The wound healing composition of claim 1, wherein the antimicrobial is selected from the group consisting of sterile saline or hydrogel, povidone-iodine solutions, cadexomer iodine, hypochlorous acid, and collagenase.

6. The wound healing composition of claim 1, wherein the composition is in a form of a patch, lotion, hydrogel, solution or cream.

7. The wound healing composition of claim 1, wherein the composition is embedded in a delivery matrix selected from the group consisting of collagen, hyaluronan, hydrogels made of Poly(vinyl alcohol) (PVA), collagen-chitosan hydrogels, alginate matrices, carbopol gels, hydrocolloidal dressing, and foam dressings.

8. The wound healing composition of claim 1, wherein the wound healing agent comprises cytokines, growth factors and/or antioxidants.

9. The wound healing composition of claim 8, wherein the growth factor comprises one or more of EGF, VEGF, TGF beta, and PDGF.

10. The wound healing composition of claim 8, wherein the antioxidant comprises alpha-tocopherol, and/or n-acetyl cysteine.

11. A biofilm treatment matrix for treating and/or preventing a wound, wherein a biofilm treatment agent comprising one or more chlorates alone or in combination with one or more antibiotics and/or antimicrobials embedded in a delivery matrix.

12. The biofilm treatment matrix of claim 11, wherein the delivery matrix is selected from the group consisting of collagen, hyaluronan, hydrogels made of Poly(vinyl alcohol) (PVA), collagen-chitosan hydrogels, alginate matrices, carbopol gels, hydrocolloidal dressing, and foam dressings.

13. The biofilm treatment matrix of claim 11, wherein the one or more chlorates are in an amount between 1 mM and 200 mM.

14. The biofilm treatment matrix of claim 11, wherein the one or more antibiotics comprises amoxicillin, clavulanic acid, clindamycin, aminoglycosides, ciprofloxacin, cefalosporines, and/or metronidazole.

15. The biofilm treatment matrix of claim 11, wherein the one or more antibiotics comprise one or more of Ciprofloxacin, Piperacillin, Ceftazidime, Aztreonam, and/or Tobramycin.

16. The biofilm treatment matrix of claim 11, wherein the antimicrobial is selected from the group consisting of sterile saline or hydrogel, povidone-iodine solutions, cadexomer iodine, hypochlorous acid, and collagenase.

17. The biofilm treatment matrix of claim 11, further comprising one or more antioxidants.

18. A method of treating a wound in an individual, the method comprising contacting the wound with the composition of claim 1, and/or with the with the composition of claim 1 within a biofilm treatment matrix.

19. The method of claim 18, further comprising before the contacting, administering a therapeutically effective amount of chlorite to the wound.

20. The method of claim 18, further comprising following contacting the chronic wound with the composition, applying to the wound a wound healing agent.

21. A method of treating a wound in an individual, the method comprising
contacting the wound with a biofilm treatment matrix of claim 11 for a time and under condition to inhibit bacteria biofilm formation and/or disrupt bacterial biofilm in the wound, and optionally
after the contacting, applying to the wound an effective amount of a wound healing agent for a time and under condition to promote re-epithelization and granulation tissue formation of the wound.

22. The method of claim 21, further comprising before the contacting, administering a therapeutically effective amount of chlorite to the wound.

23. The method of claim 21, wherein the wound is a chronic wound of the individual.

24. A method of treating a wound in an individual, the method comprising
contacting the wound of the individual with an effective amount of a biofilm treatment agent comprising a chlorate alone or in combination with an antibiotic, the contacting performed for a time and under conditions to inhibit formation of a bacteria biofilm and/or disrupt a bacteria biofilm in the wound; and
after the contacting, applying to the wound an effective amount of a wound healing agent to promote re-epithelization and granulation tissue formation of the wound.

25. The method of claim 24, further comprising before the contacting, administering a therapeutically effective amount of chlorite to the wound.

26. The method of claim 24, wherein the antibiotic comprises one or more of Ciprofloxacin, Piperacillin, Ceftazidime, Aztreonam, and Tobramycin.

27. A method of treating a wound in an individual, the method comprising
applying to the wound a wound healing agent, in combination with a biofilm treatment agent comprising a chlorate alone or in combination with an antibiotic,
wherein the wound healing agent is in an effective amount to promote re-epithelization and granulation tissue formation of the wound, and the biofilm treatment agent in an effective amount to inhibit formation of a bacteria biofilm in the wound.

28. The method of claim 27, wherein, the applying is performed after contacting the wound with an effective amount of a biofilm treatment agent comprising a chlorate alone or in combination with an antibiotic for a time and under condition to disrupt a biofilm in the wound.

29. The method of claim 27, further comprising before the contacting, administering a therapeutically effective amount of chlorite to the wound.

30. The method of claim 27, wherein the antibiotic comprises one or more of Ciprofloxacin, Piperacillin, Ceftazidime, Aztreonam, and Tobramycin.

31. A method of preventing a wound of an individual from becoming a chronic wound, the method comprising
contacting the wound with a biofilm treatment agent comprising a chlorate alone or preferably in combination with an antibiotic, the contacting performed for a time and under condition to inhibit formation of a bacteria biofilm and/or disrupt a bacteria biofilm in the wound thus preventing the wound from becoming a chronic wound.

32. The method of claim 31, further comprising applying to the wound an effective amount of a wound healing agent to promote re-epithelization and granulation tissue formation of the wound.

33. The method of claim 31, further comprising before the contacting, administering a therapeutically effective amount of chlorite to the wound.

34. The method of claim 31, wherein the antibiotic comprises one or more of Ciprofloxacin, Piperacillin, Ceftazidime, Aztreonam, and Tobramycin.

35. The method of claim 18, wherein the wound is a chronic wound.

36. The method of claim 18, wherein the one or more chlorates are in an amount between 1 mM and 200 mM.

37. The method of claim 18, wherein the one or more antibiotics comprises one or more of amoxicillin, clavulanic acid, clindamycin, aminoglycosides, ciprofloxacin, cefalosporines, and metronidazole.

38. The method of claim 18, wherein the antimicrobial is selected from the group consisting of sterile saline or hydrogel, povidone-iodine solutions, cadexomer iodine, hypochlorous acid, and collagenase.

39. A system for treating and/or preventing a wound, the system comprising:
one or more chlorates, one or more antibiotics, one or more other antimicrobials, and/or one or more wound healing agents,
for concurrent combined or sequential use in a method according to claim 18.

40. The system of claim 39, further comprising chlorite in an effective amount to be used for treating and/or preventing a wound.

41. A method to treat a systemic and/or chronic infection resulting in a wound in an individual, the method comprising
treating and/or preventing the wound in the individual by performing a method according to claim 18.

42. A system to treat a systemic and/or chronic infection resulting in a wound in an individual, the system comprising
the system of claim 41 possibly in combination with additional therapeutic agent selected to treat the systemic and/or chronic infection.


SEQUENCE LISTING (5 sequences)

SEQ ID NO: 1 - protein, 1261 aa, P. aeruginosa
  MISC_FEATURE (1)..(1261): Sequence of NarG gene
MSHLLDRLQFFKKKQGEFADGHGETSNESRAWEGAYRQRWQHDKIVRSTHGVNCTGSCSWKIYVKNGLITWETQQTDYPRTRPDLPNHEPRGCPRGASYSWYIYSANRLKYPKVRKPLLKLWREARAQHGDPVNAWASIVEDAAKAKSYKSQRGLGGFVRSSWDEVTEIIAAANVYTAKTYGPDRVIGFSPIPAMSMVSYAAGARYLSLIGGVCLSFYDWYCDLPPASPQIWGEQTDVPESADWYNSSYIIAWGSNVPQTRTPDAHFFTEVRYKGTKTVSITPDYSEVAKLTDLWLNPKQGTDAALGMAFGHVILKEFHLDRPSAYFVDYCRQYTDMPMLVLLEEHAGGAFKPTRYLRAADLADNLGQDNNPEWKTIAYDERSGGLVSPTGAIGYRWGESGKWNIAELDGRSGDQTRLQLSLLDGPEHACEVAFPYFAGQEHPHFKGVANDEVLLRRVPFREIVAADGKRLRVATVYDLQMANYSIDRGLGGDNVATSYEDADTPYTPAWQERITGVPAARATQVAREFADSADKTRGKAMVIIGAAMNHWYHMDMNYRAVINMLMMCGCIGQSGGGWAHYVGQEKLRPQTGWAPLAFGLDWSRPPRQMNGTSFFYLHSSQWRHEKLSMHEVLSPLADASRFAEHALDYNIQAERLGWLPSAPQLNRNPLRIAAEAEAAGLPVADYVVRELKSGGLRFASESPDDPQNFPRNMFIWRSNLLGSSGKGHEYMLKYLLGAKNGVMNDDLGKAGGPRPTEVDWVDDGAEGKLDLVTTLDFRMSSTCMYSDIVLPTATWYEKDDLNTSDMHPFIHPLSAATDPAWEAKSDWEIYKAIAKKFSAVAEGHLGVEQDLVTVPLLHDTPTELAQPFGGDGHDWKKGECEPMPGRNLPTLHLVERDYPNVYRKFTSLGPLLDKLGNGGKGIGWNTEKEVKLVGDLNHRVVESGVSQGRPRIDSAIDAAEVVLALAPETNGQVAVKAWEALSKITGREHAHLALPKEDEKIRFRDIQVQPRKIISSPTWSGLEDEHVSYNAGYTNVHELIPWRTITGRQQFYQDHPWMQAFGEGFVSYRPPVNTRTTEKLLNRKPNGNPEITLNWITPHQKWGIHSTYSDNLLMLTLSRGGPIIWLSEHDAAKAGIVDNDWVEVFNANGAATCRAVVSQRVKDGMVMMYHAQERIVNVPGSETTGTRGGHHNSVTRVVLKPTHMIGGYAQQAWGFNYYGTVGCNRDEFVVVRKMSKVDWLDEPRHGGLGGDALPQPLPQDI

SEQ ID NO: 2 - DNA, 3786 bp, P. aeruginosa
  misc_feature (1)..(3786): sequence of narG gene
atgagtcacctgctcgaccgcctgcagttcttcaagaagaagcagggcgaattcgccgatggccacggcgagaccagcaacgagagccgcgcctgggaaggtgcctaccggcagcgctggcagcacgacaagatcgtgcgctccacccacggggtgaactgcaccggctcctgctcctggaagatctacgtgaagaacggcctgatcacctgggaaacccagcagaccgactacccgcgcacccgtccggacctgcccaaccacgagccgcgcggctgcccgcgcggggccagctattcctggtacatctacagcgccaaccgcctgaagtacccgaaggtgcgcaagccgttgctcaagctctggcgcgaggcgcgggcgcagcacggcgacccggtgaacgcctgggccagcatcgtcgaggacgccgccaaggcgaagagctacaagagccagcgcggcctgggcggcttcgtccgttccagctgggacgaggtcaccgagatcatcgccgcggccaacgtctacaccgccaagacctacggtccggaccgggtgatcggcttctcgccgatcccggccatgtcgatggtcagctacgccgccggcgcccgctacctgtcgctgatcggcggggtctgcctgagcttctacgactggtactgcgacctgccgccggccagcccgcagatctggggcgagcagaccgacgtgccggagtcggccgactggtacaactccagctacatcatcgcctggggctccaacgtgccgcagacgcggaccccggacgcgcacttcttcaccgaggtgcgctacaagggcaccaagaccgtctccatcaccccggactattccgaggtggccaagctcaccgacctctggctcaaccccaagcagggcaccgacgccgcgctgggcatggccttcggtcacgtgatcctgaaggaattccacctcgaccggccgagcgcctacttcgtcgactactgccgccagtacaccgacatgccgatgctggtgttgctggaagaacacgccggcggcgcgttcaagccgacccgctacctgcgcgccgccgacctggcggacaacctcggccaggacaacaaccccgagtggaagaccatcgcctacgacgagcgcagcggcgggctggtctcgcccaccggcgccatcggctatcgctggggcgagtcaggcaagtggaacatcgccgagctggacggcaggagcggtgaccagacgcgcctgcaactgtcgctgctcgatggcccggaacatgcctgcgaggtggccttcccgtatttcgccgggcaggagcacccgcacttcaagggcgtcgccaacgacgaggtactgctgcgccgggtgccgttccgcgagatcgtcgcggcggacggcaagcgcctgcgggtggccaccgtctacgacctgcagatggccaactacagcatcgaccgcggcctgggcggcgacaacgtggcgacctcctacgaggacgccgacacgccctataccccggcctggcaggagcgcatcaccggcgttccggcggcgcgcgcgacgcaggtcgcccgcgagttcgccgacagcgccgacaagacccgcggcaaggcgatggtgatcatcggcgcggcgatgaaccactggtaccacatggacatgaactaccgcgcggtcatcaacatgctgatgatgtgcggctgcatcggccagagcggcggcggctgggcgcactatgtcggccaggagaagctgcgcccgcagaccggctgggcgccgctggccttcggcctggactggagccggccgccgcggcagatgaacggcaccagcttcttctacctgcacagctcgcaatggcgccacgagaagctgtcgatgcacgaggtgctgtcgccgctggccgacgccagccgcttcgccgaacacgccctggactacaacatccaggccgaacgcctcggctggctgccgtcggcgccgcaactgaaccgcaacccgctgcgcatcgccgccgaggccgaggccgccggcctgccggtcgccgactacgtggtgcgcgaactgaagagcggcggcctgcgcttcgccagcgaatcgccggacgatccgcagaacttcccgcgcaacatgttcatctggcgctccaacctgctgggctcctccggcaagggccacgagtacatgctcaagtacctgctcggggcgaagaacggggtgatgaacgatgacctcggcaaggccggcggtccgcgtcccaccgaggtcgactgggttgacgacggtgccgagggcaagctcgacctggtcaccaccctggacttccgcatgtcctccacctgcatgtactcggacatcgtcctgccgaccgctacctggtacgagaaggacgacctcaacacctccgacatgcaccccttcatccatccgctgtcggcggccaccgatccggcctgggaagccaagagcgactgggagatctacaaggccatcgccaagaagttctccgccgtcgccgaaggccacctcggcgtggagcaggacctggtcacggtgccgctgctgcacgacacccccaccgagctggcgcagccgttcggcggcgacggccatgactggaagaagggcgagtgcgagccgatgccgggacgcaacctgccgacgctgcacctggtcgagcgcgactacccgaacgtctaccgcaagttcacctcgctcggtccgctgctggacaagctgggcaacggcggcaagggcatcggctggaacaccgagaaggaagtgaagctggtcggcgacctcaaccatcgcgtcgtcgagagcggcgtcagccagggccgcccgcgcatcgacagcgccatcgacgccgctgaggtggtcctcgccctggctccggaaaccaacggccaggtcgcggtcaaggcctgggaagcgctgtcgaagatcaccggccgcgagcatgcccacctggcgctgcccaaggaagacgagaagatccgcttccgcgacatccaggtgcagccgcgcaagatcatctccagcccgacctggtccggcctcgaggacgagcacgtcagctacaacgccggctacaccaacgtccacgagctgatcccgtggcgcaccatcaccggtcgccagcagttctaccaggaccacccgtggatgcaggcgttcggcgaaggcttcgtcagctaccggccgccggtcaacacccggaccaccgagaaactgttgaacaggaagcccaacggcaacccggagatcaccctgaactggatcaccccgcaccagaaatggggcatccactccacctacagcgacaacctgctgatgctcaccctgtcgcgcggcggtccgatcatctggctcagcgagcacgacgcggccaaggccgggatcgtcgataacgactgggtcgaggtgttcaacgccaacggcgcggcgacctgccgcgcggtggtcagccagcgggtcaaggacggcatggtgatgatgtaccacgcccaggaacgcatcgtgaacgtacccggcagcgagaccaccggcacccgtggcggccaccacaactcggtgacccgcgtggtgctcaagccgacccacatgatcggcggctacgcccagcaggcctggggcttcaactactacggcacggtcggctgcaaccgcgacgagttcgtcgtggtgcgcaagatgagcaaggtcgactggctggacgaaccccgccacggcggactcggcggcgacgccctgccgcaaccgctgccccaggacatttga

SEQ ID NO: 3 - DNA, 1542 bp, P. aeruginosa
  misc_feature (1)..(1542): sequence of narH gene
atgaaaattcgttcgcaagtcggcatggtgctgaacctcgacaagtgcattggttgccacacctgctcgatcacctgcaagaacgtctggaccagccgcgaaggcatggagtacgcctggttcaacaacgtcgagaccaagcccgggatcggctacccgaaggaatgggaaaaccaggagaagtggaagggcggctgggtgcgcgcggcggacggttcgatccgcccgcgcatcggcggcaagttccgcgtgctggcgaacatcttcgccaacccggacctgcccgagatcgacgactactacgaaccgttcgacttcgattaccagcacctgcataccgcgcccaaggccgagcaccagccggtggcgcgcccgcgctcgctggtctccgggcagcgcatggagaagatcgagtggggcccgaactgggaggagatcctcggcaccgagttcgccaagcggcgcaaggacaagaacttcgaccaggtccaggcggacatctacggtgagtacgagaacaccttcatgatgtacctgccgcgcctctgcgagcactgcctgaacccggcgtgcgtggcgtcctgcccgagcggggcgatctacaagcgcgaggaggacggcatcgtcctgatcgaccaggacaagtgccgcggctggcggatgtgcatctccggctgcccgtacaagaagatctacttcaactggaagagcggcaagtccgagaagtgcatcttctgctacccgcgcatcgaggccggccagcccactgtctgctcggagacctgcgtcgggcgcatccgctacctcggcgtgctgctctacgacgccgaccgcatccacgaagtggccagttgcgagaacgagcgcgagctgtacgagaagcaactggagatcttcctcgatccgttcgacccggcggtgatcgcccaggcgcgcaaggacggggtggccgacagcgtcatcgaggcggcgcagaagtcgccggtgtacaagctggcgatggactggaagctggccctgccgctgcacccggaataccgcacgctgccgatggtctggtacgtgccaccgctgtcgccgatccagaacgccgccgccgaggggcacatcggcagcgacggggtgatcccggacgtggagtcgctgcgcatccccgtgcagtacctggccaacctgctcaccgccggcgacaccgcgccggtgctgctggcgctcaagcgcctgctggcgatgcgcgcctacaagcgcgccgagcacgtcgaaggccgccaggacctggaggtgctggccaaggtcgggttgagcgtggagcaggtggaggagatgtaccgctacctggccatcgccaactacgaggatcgcttcgtgatccccagcgcgcaccgcgaggaagcgctttccgatgccttcgccgagcgttccggctgcggcttcagcttcggcaacggctgttccggcggcagcaactccgccgtcaacctgttcggcggcaagccgaccaaccgccgcgacgtgatccaggtcgtgcagatccaggagtga

SEQ ID NO: 4 - DNA, 741 bp, P. aeruginosa
  misc_feature (1)..(741): sequence of narJ gene
atgaacgatcacagccaactgttccgcctgctcgccctgctgctcgactatccacgcgccgagctgcgcgaggagagcctcggcctgcatgcgctgatccgcacctgcgaactgccggaagcgctgcgcgacggcctcgcggcgctgctcaacgagctctgccagggcgacctgctggacgtccaggcgcgctacgacggtctcttcgagcgcggccgctcggtctcgctgctgctcttcgagcacgtccacggcgagagccgcgaccgtggccaggcgatggtcgacctgctcgaccgctataccggggccggcctgcagatcgacgtaccggagctgccggactacctgccgctgtacctcgaatacctgtcgctgctgccgttcgcggcggccagcgaagggctcgccgaagtggcgcacatcctcggcctgctggcgctgcgcctggaggaacgcggcagcgcctacgcggcgattttcgaggcgttgctggaactcggcggcgagcgcccggacctcggcgcgttgcgtcgcgaccaggcccaggaacagcgcgacgacagcctggaggccatcgaccgggcctgggaggaaaccccggtgagcttcaccgaccctgccggcggttgcccgtcgagcagcggccgccgtccgacggcgtccaccgaacaaccattgcaatgggtcgcccagccggtaccgcagatgcagtaccgcgcggcccgcgaaggagtctga

SEQ ID NO: 5 - DNA, 684 bp, P. aeruginosa
  misc_feature (1)..(684): sequence of narI gene
atgtcgaccaatcttctgttcttcgggatctatccctatgtcgcgctgctgatctgcctggtcggcagctgggcgcgcttcgacctctcgcagtacacctggaaggccggttccagccagatgctcagcaagaagggcatgcgggtatacagcaacctgttccacgtcggcgtgctgttcatcctcgccggccacttcgtcggcctgctgaccccggcctcggtctaccaccacctgatcagcaccgagaacaagcaactgctggcgatggtctccggcggcttcttcggcgtgctctgcttcatcggcctgagcggactgatcctgcgccgcctgaccgacgcgcgggtgcgcgccaccggcaacgcctctgacctgatgatcctgctggtgctctacgcccagctgatcctcggcctctccaccatcgtcgcctcgacccatcacatggatggctcggtgatggtgatgctcgccgactgggcccaggccatcgtcaccctgcgtccgctggcggcggccgaagccatcgcgccggtgggcctggtctacaagctgcacgtcggcctgggcctgaccctgttcgtgctgttccccttcacccgcctggtgcacatcgtcagcgcgccggtgtggtacctgggccggcgctaccagatcgtgcgtcagaaacgtcctgcctga